(19) United States
(12) Patent Application Publication
(10) Pub. No.: US 2026/0199368 A1
(43) Pub. Date: Jul. 16, 2026

(54) OROMUCOSAL DELIVERY SYSTEM CONTAINING REMIMAZOLAM

(71) Applicants: LTS LOHMANN THERAPIE-SYSTEME AG, Andernach (DE); PAION UK LTD., Richmond, London (GB)

(72) Inventors: Stefan LORSCHEIDT, Bonn (DE); Eva-Marie PRINZ, Weißenthurm (DE); Elke KLEIN, Bad Neuenahr-Ahrweiler (DE); Marco EMGENBROICH, Rheinbach (DE); Marija PESIC, Aachen (DE); Oliver KOPS, Aachen (DE); Thomas STÖHR, Mol (BE)

(21) Appl. No.: 19/136,442

(22) PCT Filed: Dec. 14, 2023
(86) PCT No.: PCT/EP2023/085948

§ 371 Date: Jun. 6, 2025

(30) Foreign Application Priority Data
Dec. 16, 2022 (EP) 22214391.9

(51) Int. Cl. A61K 31/5517; A61K 9/00; A61K 47/10; A61K 47/34; A61K 47/38; B65D 75/20; B65D 81/26

(52) U.S. Cl. CPC A61K 31/5517; A61K 9/006; A61K 47/10; A61K 47/34; A61K 47/38; B65D 75/20; B65D 81/267

(57) ABSTRACT

The present invention relates to oromucosal delivery systems for the transmucosal administration of an active agent comprising an active agent-containing layer comprising remimazolam, such remimazolam oromucosal delivery systems for producing sedation, a method of producing sedation comprising applying such remimazolam oromucosal delivery systems, and processes of manufacture of such remimazolam oromucosal delivery systems.

### TECHNICAL FIELD OF THE INVENTION

The present invention relates to an oromucosal delivery system for the transmucosal administration of remimazolam to the systemic circulation, and process of manufacture, methods of treatment and uses thereof.

### BACKGROUND OF THE INVENTION

Remimazolam (methyl 3-{(4S)-8-bromo-1-methyl-6-(pyridin-2-yl)-4/-/-imidazo[1,2-a][1,4]benzodiazepin-4-yl}propanoate) is a novel benzodiazepine sedative identified as one of the lead compounds in a program focusing on ester-based benzodiazepine derivatives with a short and predictable duration of action initiated in the late 1990's.

Remimazolam comprises an ester group which is rapidly hydrolyzed by tissue esterases (Carboxylesterase 1) and transformed into the inactive metabolite CNS7054, thus making it a very short-acting drug.

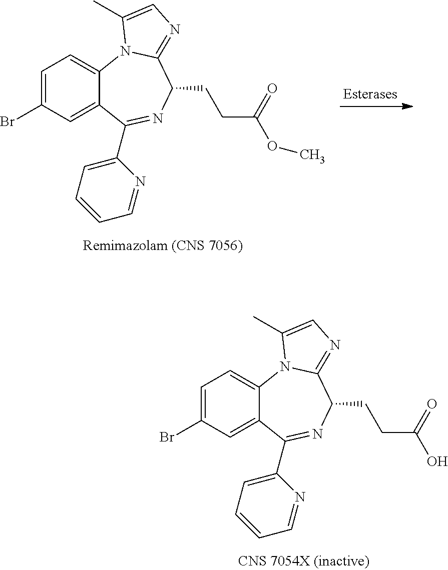 [SMILES: COC(=O)CC[C@@H]1N=C(c2ccccn2)c2cc(Br)ccc2-n2c(C)cnc21 ; Cc1cnc2n1-c1ccc(Br)cc1C(c1ccccn1)=N[C@H]2CCC(=O)O]

It exhibits anxiolytic, amnestic, sedative, muscle relaxing, and anticonvulsant properties. Due to these properties, it is suitable for use in anesthetic practice and intensive care, such as in preoperative sedation, anxiolysis, amnestic use for perioperative cases, conscious sedation during short diagnostic, operative, or endoscopic procedures, for example, as a component for inducing and maintaining general anesthesia, before and/or along with the administration of other anesthetic agents, as well as in intensive care sedation.

Two salt forms have been developed in particular: besylate and tosylate. Remimazolam besylate has been approved for general anesthesia in Japan and South Korea as well as for procedural sedation in the United States, China and Europe. The tosylate salt has been approved for procedural sedation in China.

Remimazolam besylate is produced in lyophilized form for reconstitution. There is currently no ready-to-use formulation available.

In terms of administration routes, the oral bioavailability of remimazolam is poor (1~2% having been reported) because of extensive carboxylesterase-mediated first pass metabolic elimination in the liver. Intranasal administration achieves a significantly higher bioavailability (about 50%), however, may be associated with nasal discomfort/pain in certain cases. Inhalation has been proposed as an alternative.

The primary route considered for remimazolam thus remains intravenous (IV) administration so far. In Europe, remimazolam besylate has been approved as Byfavo, 20 mg powder for solution for injection. In certain settings, however, alternatives to the IV route would be desired. The lyophilized form of the drug product requires reconstitution of the active before IV administration. Strict hygienic conditions are required for preparing injection and special care should be taken to dispose needles. In particular younger patients fear pain associated with injections.

It would therefore be desirable to provide alternative, ready-to-use non-invasive remimazolam dosage forms (wherein it is understood that this includes remimazolam, a pharmaceutically acceptable salt, or any other form thereof). Remimazolam, however, is prone to degradation by hydrolysis and developing formulations with sufficient stability resulting in an appropriate shelf-life is challenging. In addition, due to the low bioavailability as outlined above, the classical oral route, for which extensive knowledge on formulation technology would be available, does not seem to be useful, and developing an appropriate non-oral drug delivery route which is patient-friendly, has a fast onset of action and provides appropriate pharmacokinetic characteristics can be regarded as a highly demanding task.

It is therefore unsurprising that up to date, no viable remimazolam dosage form other than the IV formulation appears to be available, and no ongoing/recent research or investigation on alternative remimazolam dosage forms except those mentioned above is known to the Applicant.

There is thus a need for an alternative dosage form of remimazolam which overcomes the disadvantages of low bioavailability associated with oral administration as well as the disadvantages related to intravenous administration.

### OBJECTS AND SUMMARY OF THE INVENTION

It is an object of the present invention to provide a remimazolam dosage form overcoming one or more of the above-mentioned disadvantages of current remimazolam administration.

It is an alternative object of the present invention to provide a remimazolam dosage form which is non-invasive, patient-friendly and/or based on a ready-to-use formulation.

It is also an alternative object of the present invention to provide a remimazolam dosage form which provides high bioavailability, in particular when compared to oral administration.

It is a further alternative object of the present invention to provide a remimazolam dosage form providing a fast onset of action and/or a reliable onset of action, in particular when compared to oral administration.

It is also a further alternative object of the present invention to provide a remimazolam dosage form providing a high permeation rate which is sufficient for achieving a therapeutically effective dose of remimazolam.

It is a yet further alternative object of the present invention to provide a remimazolam dosage form providing an extended duration of action as compared to a single dose of IV remimazolam.

It is another alternative object of the present invention to provide a remimazolam dosage form wherein remimazolam is stable and which can be stored, e.g., at room temperature.

It is also another alternative object of the present invention to provide a remimazolam dosage form which does not provoke an irritating sensation at the administration site.

It is yet another alternative object of the present invention to provide a remimazolam dosage form which complies with the needs of a convenient application and handling, provides for good patient compliance and/or which is easy and cost-efficient to manufacture.

One or more of the above objects and others is/are accomplished by the present invention, which according to one aspect relates to an oromucosal delivery system for the transmucosal delivery of an active agent, comprising an active agent-containing layer comprising
i) remimazolam, a pharmaceutically acceptable salt, or any other form thereof, as active agent and
ii) a film-forming agent.

According to another aspect of the invention, the oromucosal delivery system according to the invention is for use in producing sedation, producing hypnosis, producing anxiolysis, producing muscle relaxation, the treatment of convulsions or the induction of amnesia for perioperative events.

According to another aspect, the present invention also relates to a method of producing sedation, producing hypnosis, producing anxiolysis, producing muscle relaxation, treating convulsions or inducing amnesia for perioperative events, wherein the oromucosal delivery system is administered to a subject.

According to another aspect, the present invention also relates to the use of the oromucosal delivery system in the preparation of a medicament for producing sedation, producing hypnosis, producing anxiolysis, producing muscle relaxation, the treatment of convulsions or the induction of amnesia for perioperative events.

According to yet another aspect, the present invention relates to a pharmaceutical product comprising a packaging and one or more unit doses of the oromucosal delivery system.

According to a further aspect, the invention relates to a process of manufacture of an active agent-containing layer comprising the steps of:
i. combining at least (i) remimazolam, a pharmaceutically acceptable salt, or any other form thereof, as active agent, and (ii) a film-forming agent to obtain a mixture; and
ii. Forming the active agent-containing layer.

According to a further aspect, the invention relates to a process of manufacture of an oromucosal delivery system comprising an active agent-containing layer comprising (i) remimazolam, a pharmaceutically acceptable salt, or any other form thereof, as active agent, and (ii) a film-forming agent comprising the steps of:
i. Combining at least the active agent and a film-forming agent to obtain a mixture; and
ii. Forming the active agent-containing layer.

According to a further aspect, the invention relates to an oromucosal delivery system obtainable by such a process.

According to certain embodiments, the invention also relates to an oromucosal delivery system for the transmucosal administration of remimazolam comprising an active agent-containing layer comprising
i) 55 to 60 wt-% of remimazolam besylate;
ii) 10 to 15 wt-% of polyvinyl alcohol as film-forming agent,
iii) 30 to 35 wt-% of a polyvinyl alcohol-polyethylene glycol grafted copolymer,
iv) from 0.05 to 1 wt-% of one or more sweeteners, and
v) from 0.5 to 2 wt-% of a flavoring agent
   wherein
   the area weight of the active agent-containing layer is less than or equal to 200 g/m².

According to certain embodiments, the invention also relates to an oromucosal delivery system for the transmucosal administration of remimazolam comprising an active agent-containing layer comprising
i) 55 to 60 wt-% of remimazolam besylate;
ii) 15 to 20 wt-% of polyvinyl alcohol as film-forming agent
iii) 20 to 26 wt-% of a polyvinyl alcohol-polyethylene glycol grafted copolymer,
iv) from 0.05 to 1 wt-% of one or more sweeteners, and
v) from 0.5 to 2 wt-% of a flavoring agent
   wherein
   the area weight of the active agent-containing layer is less than or equal to 200 g/m².

Within the meaning of this invention, the term “oromucosal delivery system” refers to a system by which the active agent (remimazolam, a pharmaceutically acceptable salt, or any other form thereof) is administered to the systemic circulation via transmucosal delivery by application to the mucosa of the oral cavity, and refers to the entire individual dosing unit that is applied to the mucosa of a patient, and which in particular comprises a therapeutically effective amount of remimazolam, a pharmaceutically acceptable salt, or any other form thereof in an active agent-containing layer. Oromucosal delivery systems consist of one or more thin layers which are applied and adhere to the mucosa of the oral cavity to deliver the active agent. Dosage forms in the form of thin films for application in the oral cavity are also sometimes referred to as “Oral Thin Film” or OTF, however, OTFs are not necessarily intended to adhere to the mucosa. In an oromucosal delivery system, the active agent is contained in a dissolvable layer, and due to the film adhering to the mucosa, active delivery is primarily achieved by a combination of local active release from the oromucosal delivery system to the mucosa (i.e., the active is dissolved from the system and released directly into the mucosa underneath), and by an “indirect” active delivery due to the active, once dissolved in the saliva, migrating from the administration site to other parts of the oral cavity.

Within the meaning of this invention, the term “oromucosal delivery system” in particular refers to a system which is mucoadhesive and provides for passive transmucosal delivery excluding active transport as in methods including microporation. Also, in contrast to certain oral thin films which are not necessarily mucoadhesive and which are intended to disintegrate very fast in the saliva (sometimes referred to as “flash wafers”), enteral delivery is unintended in oromucosal delivery systems, albeit not excluded (e.g., by unintentional swallowing of saliva).

Within the meaning of this invention, the term “active agent-containing layer” refers to the layer comprising the active agent and a film-forming agent, which will form a matrix with the active that disintegrates upon contact with saliva and releases the active. The active agent-containing layer may be mucoadhesive (in the form of a mucoadhesive layer) or the oromucosal delivery system may comprise an additional mucosa-contacting layer of a mucoadhesive for providing sufficient adhesion. In particular, the active agent-containing layer is a mucoadhesive layer.

Within the meaning of this invention, the term “therapeutically effective amount” refers to a quantity of active agent in the oromucosal delivery system sufficient to provide, upon administering the oromucosal delivery system to a patient, sedation and/or remimazolam blood levels of a similar range (e.g. overall maximum plasma concentration cₘₐₓ of 189 to 6,960 ng/mL) when compared to the sedation and/or blood levels obtained after a one-time IV administration of 0.01 to 0.5 mg/kg remimazolam, a pharmaceutically acceptable salt, or any other form thereof.

Within the meaning of this invention, the terms “active”, “active agent”, and the like, refer to remimazolam as well as to a pharmaceutically acceptable salt or any other form thereof, and in particular to any pharmaceutically acceptable chemical and morphological form and physical state thereof. These forms include without limitation remimazolam (the free base form), protonated or partially protonated remimazolam, remimazolam salts and in particular acid addition salts formed by addition of an inorganic or organic acid such as remimazolam besylate or remimazolam tosylate, hydrates, solvates, complexes and so on, as well as the active agent in the form of particles which may be micronized or may preferably not be micronized, crystalline and/or amorphous, and any mixtures of the aforementioned forms. The active agent, where contained in a medium such as a solvent, may be dissolved or dispersed or in part dissolved and in part dispersed.

When the active agent is mentioned to be used in a particular form in the manufacture of the oromucosal delivery system, this does not exclude interactions between this form of the active agent and other ingredients of the active agent-containing self-adhesive layer structure, e.g. salt formation or complexation, in the final oromucosal delivery system. This means that, even if the active agent is included in its free base form, it may be present in the final oromucosal delivery system in protonated or partially protonated form or in the form of an acid addition salt, or, if it is included in the form of a salt, parts of it may be present as free base in the final oromucosal delivery system. Unless otherwise indicated, the amounts given for the active agent are calculated based on remimazolam in the form of the free base. In particular the amount of active agent in the active agent-containing layer relates to the amount of the active agent included in the oromucosal delivery system during manufacture of the oromucosal delivery system and is calculated based on remimazolam in the form of the free base. E.g., when a) 0.1 mmol (equal to 43.9 mg) remimazolam base or b) 0.1 mmol (equal to 59.8 mg) remimazolam besylate is included in the oromucosal delivery system during manufacture, the amount of active agent in the self-adhesive layer structure is given, within the meaning of the invention, in both cases, as 0.1 mmol or 43.9 mg.

The active agent starting material included in the oromucosal delivery system during manufacture of the oromucosal delivery system may be in the form of particles. The active agent may e.g. be present in the mucoadhesive layer structure in the form of particles, dispersed and/or dissolved.

Within the meaning of this invention, the term “particles” refers to a solid, particulate material comprising individual particles, the dimensions of which are negligible compared to the material. In particular, the particles are solid, including plastic/deformable solids, including amorphous and crystalline materials.

Within the meaning of this invention, the term “dispersing” refers to a step or a combination of steps wherein a starting material (e.g. remimazolam) is not totally dissolved. Dispersing in the sense of the invention comprises the dissolution of a part of the starting material (e.g. remimazolam particles), depending on the solubility of the starting material (e.g. the solubility of remimazolam in the coating composition).

Terms such as “dissolution”, “dissolvable”, “dissolve” and the like with respect to the active agent-containing layer of an oromucosal delivery system and with respect to the film-forming agent when casted into a film, are to be understood very broadly, and not in the strict scientific sense of chemically dissolving a molecule in a solvent. Any transformation of the solid state of the layer concerned to a liquid state, such as dispersing, forming of a suspension, gelling of the film and disintegrating into smaller parts of gel, etc. has to be regarded as “dissolving” in the sense of the present invention, as long as the “dissolved” material is able to freely move around in the liquid (e.g. saliva). In preferred embodiments, the meaning is limited to the usual chemical sense of dissolving a molecule in a solvent. It should be noted that the term “dissolve” with respect to substances per se, such as the active agent remimazolam, or any excipients, will continue to be used in the usual chemical sense of dissolving a molecule in a solvent. E.g., remimazolam in dissolved form obviously does not include remimazolam in dispersed form. The film-forming agent per se can be present in the coating composition during manufacture of the oromucosal delivery system in dissolved form in the common chemical sense (e.g. is not dispersed, in form of small parts of gel, etc.), but where the film-forming agent is casted into a film, “dissolving” such a film also includes gelling of the film and disintegrating into smaller parts of gel.

Within the meaning of this invention, the term “mucoadhesive” refers to a material that in particular adheres to and upon contact with a mucosa, but which preferably is non-tacky and can be touched e.g. with the fingers and manipulated, e.g. for application into the oral cavity, without unintentionally adhering to the skin of the fingers, when in dry state. A mucoadhesive layer, when in contact with the mucosa, is “self-adhesive”, i.e. provides adhesion to the mucosa so that typically no further aid for fixation is needed. The adhesion strength is preferably strong enough that typical movements in the oral cavity are not sufficient to displace a mucoadhesive layer adhered to the mucosa.

Within the meaning of this invention, the term “area weight” refers to the dry weight of a specific layer, e.g. of the active agent-containing layer, provided in g/m². The area weight values are subject to a tolerance of ±10%, preferably ±7.5%, due to manufacturing variability.

If not indicated otherwise “%” refers to weight-%.

Within the meaning of this invention, the term “polymer” refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2,000, preferably above 5,000 and more preferably above 10,000 Dalton. Correspondingly, compounds with a molecular weight below 2,000, preferably below 5,000 or more preferably below 10,000 Dalton are usually referred to as oligomers.

The oromucosal delivery systems according to the present invention can be characterized by certain parameters as measured in an in vitro permeation test.

The in vitro permeation test is performed with human or animal mucosa and preferably with dermatomed split-thickness pig mucosa with a thickness of 400 μm and an intact barrier function, and with phosphate buffer pH 5.5 or 7.4 as receptor medium (37° C.) with or without addition of a maximum of 40 vol-% organic solvent e.g. ethanol, acetonitrile, isopropanol, dipropylene glycol, PEG 400 so that a receptor medium may e.g. contain 60 vol-% phosphate buffer pH 5.5, 30 vol-% dipropylene glycol and 10 vol-% acetonitrile.

Where not otherwise indicated, the in vitro permeation test is performed with dermatomed split-thickness pig mucosa (mucosa oesophagus) with a thickness of 400 μm and an intact barrier function, and with phosphate buffer pH 7.4 as receptor medium (37° C.). The amount of active permeated into the receptor medium is determined in regular intervals using an HPLC method with a UV photometric detector by taking a sample volume. The measured amount of active permeated relates to the amount permeated between the two last sampling points and not the total amount permeated so far. Within the meaning of this invention, artificial saliva refers to an aqueous solution of 0.520 g/L potassium thiocyanate, 1.470 g/L potassium chloride, 0.190 g/L sodium dihydrogenphosphate monohydrate and 2.650 g/L sodium hydrogenphosphate dihydrate, adjusted to a pH of 7.0+/−0.05 using 1 N NaOH.

Thus, within the meaning of this invention, the parameter “permeated amount” is provided in μg/cm² and relates to the amount of active permeated in a sample interval at certain elapsed time per area of release. E.g., in an in vitro permeation test as described above, wherein the amount of active permeated into the receptor medium has been e.g. measured at minutes 0, 2, 4, 8, 12 and 24, the “permeated amount” of active can be given e.g. for the sample interval from minute 8 to minute 12 and corresponds to the measurement at minute 12.

The permeated amount can also be given as a “cumulative permeated amount”, corresponding to the cumulated amount of active permeated at a certain point in time. E.g., in an in vitro permeation test as described above, wherein the amount of active permeated into the receptor medium has been e.g. measured at minutes 0, 2, 4, 8, 12 and 24, the “cumulative permeated amount” of active at minute 12 corresponds to the sum of the permeated amounts from minute 0 to minute 2, minute 2 to minute 4, minute 4 to minute 8 and minute 8 to minute 12.

Within the meaning of this invention, the parameter “mucosa permeation rate” for a certain sample interval at certain elapsed time is provided in μg/(cm² min) and is calculated from the permeated amount in said sample interval as measured by in vitro permeation test as described above in μg/cm², divided by the minutes of said sample interval. E.g. the mucosa permeation rate in an in vitro permeation test as described above, wherein the amount of active permeated into the receptor medium has been e.g. measured at minutes 0, 2, 4, 8, 12 and 24, the “mucosa permeation rate” at minute 12 is calculated as the permeated amount in the sample interval from minute 8 to minute 12 divided by 4 minutes.

A “cumulative mucosa permeation rate” can be calculated from the respective cumulative permeated amount by dividing the cumulative permeated amount by the elapsed time. E.g. in an in vitro permeation test as described above, wherein the amount of active permeated into the receptor medium has been e.g. measured at minutes 0, 2, 4, 8, 12 and 24, the “cumulative mucosa permeation rate” at minute 12 is calculated as the cumulative permeated amount for minute 12 (see above) divided by 12 minutes.

Within the meaning of this invention, the above parameters permeated amount and mucosa permeation rate (as well as cumulative permeated amount and cumulative mucosa permeation rate) refer to mean values calculated from 3 in vitro permeation test experiments.

The oromucosal delivery system according to the present invention can also be characterized by certain parameters as measured in an in vivo non-clinical or clinical study.

Within the meaning of this invention, the term “administration” refers to the application of the dosage form, i.e. the oromucosal delivery system, to the oral mucosa of the patient, which is then maintained on the mucosa until the active agent-containing layer structure is dissolved.

Within the meaning of this invention, the term “room temperature” refers to the unmodified temperature found indoors in the laboratory where the experiments are conducted and usually lies within 15 to 35° C., preferably about 18 to 25° C.

Within the meaning of this invention, the term “patient” refers to a subject who has presented a clinical manifestation of a particular symptom or symptoms suggesting the need for treatment, or for a procedure, in particular requiring sedation, who is treated preventatively or prophylactically for a condition, or who has been diagnosed with a condition to be treated which requires sedation.

Within the meaning of this invention the term “pharmacokinetic parameters” refers to parameters describing the blood plasma curve, e.g. Cₘₐₓ, Cₜ and AUCₜ₁₋ₜ₂ obtained in a clinical study, e.g. by single-dose or multi-dose administration of the oromucosal delivery system to healthy human subjects. The pharmacokinetic parameters of the individual subjects are summarized using arithmetic and geometric means, e.g. a mean Cₘₐₓ, a mean AUCₜ and a mean AUC_{INF}, and additional statistics such as the respective standard deviations and standard errors, the minimum value, the maximum value, and the middle value when the list of values is ranked (Median). In the context of the present invention, pharmacokinetic parameters, e.g. the Cₘₐₓ, Cₜ and AUCₜ₁₋ₜ₂ refer to arithmetic or geometric mean values and preferably refer to geometric mean values. It cannot be precluded that the absolute mean values obtained for a certain oromucosal delivery system in a clinical study vary to a certain extent from study to study. To allow a comparison of absolute mean values between studies, a reference formulation, e.g. in the future any product based on the invention, may be used as internal standard. A comparison of the AUC per area of release of the respective reference product in the earlier and later study can be used to obtain a correction factor to take into account differences from study to study.

Within the meaning of this invention, the parameter “AUC” corresponds to the area under the plasma concentration-time curve. The AUC value is proportional to the amount of active agent absorbed into the blood circulation in total and is hence a measure for the bioavailability.

Within the meaning of this invention, the parameter “AUCₜ₁₋ₜ₂” is provided in (ng/ml) h and relates to the area under the plasma concentration-time curve from hour t1 to t2 and is calculated by the linear trapezoidal method.

Within the meaning of this invention, the parameter “Cₘₐₓ” is provided in (ng/ml) and relates to the maximum observed blood plasma concentration of the active agent.

Within the meaning of this invention, the parameter “Cₜ” is provided in (ng/ml) and relates to the blood plasma concentration of the active agent observed at hour t.

Within the meaning of this invention, the parameter “tₘₐₓ” is provided in h and relates to the time point at which the Cₘₐₓ value is reached. In other words, tₘₐₓ is the time point of the maximum observed plasma concentration.

Within the meaning of this invention, the term “mean plasma concentration” is provided in (ng/ml) and is a mean of the individual plasma concentrations of active agent, e.g. remimazolam, at each point in time. Within the meaning of this invention, “bioavailability” is provided in %, i.e., refers to the dose normalized ratio of either Cₘₐₓ following IV administration vs dose normalized Cₘₐₓ following oromucosal administration or of dose normalized AUC following IV administration vs dose normalized AUC following oromucosal administration.

Within the meaning of this invention, the term “coating composition” refers to a composition comprising all components of the active agent-containing layer in a solvent.

Within the meaning of this invention, the term “dissolve” in the context of the preparation of the coating composition, e.g. dissolving components of the coating composition such as the active agent, refers to the process of obtaining a solution, which is clear and does not contain any particles, as visible to the naked eye.

Within the meaning of this invention, the term “foam” refers to a state of liquid or solid material entrapping relatively large amounts of gas pockets, separated by relatively thin layers of the material. A “monolithic film” on the contrary refers to a uniform film composed of one continuous phase substantially without large amounts of pores or trapped gas pockets.

Within the meaning of this invention, the term “desiccant” refers to a hygroscopic material that absorbs or adsorbs water molecules form the surrounding air and by this decreasing the level of moisture.

Within the meaning of this invention, the term “solvent” refers to any liquid substance, which preferably is water or a volatile organic liquid such as methanol, ethanol, isopropanol, acetone, ethyl acetate, methylene chloride, hexane, n-heptane, heptanes, toluene and mixtures thereof.

Within the meaning of this invention, and unless otherwise specified, the term “about” refers to an amount that is ±10% of the disclosed amount. In some embodiments, the term “about” refers to an amount that is ±5% of the disclosed amount. In some embodiments, the term “about” refers to an amount that is ±2% of the disclosed amount.

### BRIEF DESCRIPTION OF THE DRAWINGS

FIG. 1 depicts a cross section of an inventive pharmaceutical product comprising a unit dose of an inventive oromucosal delivery system in a pouch along with a folded polyethylene terephthalate foil within the primary packaging.

FIG. 2a depicts the level of sedation as determined during the in vivo studies for the placebo oromucosal delivery systems.

FIG. 2b depicts the level of sedation as determined during the in vivo studies for the oromucosal delivery system prepared according to Example 1c.

FIG. 2c depicts the level of sedation as determined during the in vivo studies for the oromucosal delivery system prepared according to Example 1d.

FIG. 2d depicts the level of sedation as determined during the in vivo studies after IV administration of remimazolam besylate.

FIG. 3a depicts the remimazolam blood plasma concentrations obtained in the in vivo studies for the oromucosal delivery system prepared according to Example 1c.

FIG. 3b depicts the CNS7054 blood plasma concentrations obtained in the in vivo studies for the oromucosal delivery system prepared according to Example 1c.

FIG. 4a depicts the remimazolam blood plasma concentrations obtained in the in vivo studies for the oromucosal delivery system prepared according to Example 1d.

FIG. 4b depicts the CNS7054 blood plasma concentrations obtained in the in vivo studies for the oromucosal delivery system prepared according to Example 1d.

FIG. 5a depicts the remimazolam blood plasma concentrations obtained in the in vivo studies after IV administration of remimazolam besylate.

FIG. 5b depicts the CNS7054 blood plasma concentrations obtained in the in vivo studies after IV administration of remimazolam besylate.

FIG. 6a depicts the remimazolam mucosa permeation rates of an oromucosal delivery system prepared according to Examples 2a when dissolved in artificial saliva or in 0.9% NaCl, or of remimazolam besylate when dissolved in artificial saliva and adjusted to a pH of 2, 3, 4.5 or 6.0, respectively.

FIG. 6b depicts the remimazolam mucosa permeation rates of an oromucosal delivery system prepared according to Examples 2a when dissolved in artificial saliva or in 0.9% NaCl, or of a remimazolam besylate lyophilizate product when dissolved in artificial saliva or in 0.9% NaCl, or of remimazolam besylate when dissolved in artificial saliva.

### DETAILED DESCRIPTION

### Oromucosal Delivery System

The present invention is related to an oromucosal delivery system for the transmucosal administration of an active agent comprising an active agent-containing layer containing remimazolam, a pharmaceutically acceptable salt, or any other form thereof, as active agent.

The active agent-containing layer comprises i) remimazolam, a pharmaceutically acceptable salt, or any other form thereof, as active agent, and ii) a film-forming agent.

Thus, the oromucosal delivery system for the transmucosal administration of an active agent comprises an active agent-containing layer comprising:
i) remimazolam, a pharmaceutically acceptable salt, or any other form thereof, as active agent, and
ii) a film-forming agent.

The oromucosal administration route is a relatively new form of drug delivery, meaning that knowledge on formulation technology is limited. Formulating appropriate dosage forms for the transmucosal delivery by oromucosal dosage forms thus is challenging. As has been explained further above, the inventive oromucosal delivery systems have been surprisingly shown to provide for an appropriate transmucosal drug delivery, thus avoiding the first-pass effect associated with enteral delivery and achieving a high bioavailability, as demonstrated by preclinical results shown further below.

Oromucosal dosage forms are non-invasive and simple and ready to use, and in this regard, address the needs and shortcomings of the IV formulation.

As outlined also above, the oromucosal delivery system consists of one or more thin layers, thus, in certain embodiments, the oromucosal delivery system is in the form of a film. Such a film may have a circular, rectangular or square shape, or any other shape.

The film has a certain degree of thickness, as otherwise it will be difficult to incorporate the required amount of active, and as very thin films are not easy to manufacture, in particular with respect to providing an even thickness. Thus, in certain embodiments, the oromucosal delivery system is in the form of a thin film wherein the film has an area weight of at least 100 g/m², at least 110 g/m² or at least 120 g/m² or has an area weight of less than or equal to 400 g/m², less than or equal to 300 g/m² or less than or equal to 250 g/m², and/or wherein the film has an area weight from 100 g/m² to less than or equal to 230 g/m² or from 300 g/m² to less than or equal to 400 g/m².

In certain embodiments of the present invention, the oromucosal delivery system is in the form of a film wherein the film has a size of at least 0.5 cm², or less than or equal to 10 cm², or of about 1.5 cm², about 3 cm² or about 6 cm².

In some embodiments, the oromucosal delivery system for the transmucosal administration of an active agent according to this invention does not contain preservatives.

### Active Agent-Containing Layer

As outlined in more detail above, the oromucosal delivery system according to the present invention comprises an active agent-containing layer comprising remimazolam, a pharmaceutically acceptable salt, or any other form thereof, as active agent, and a film-forming agent.

As outlined also above and without wishing to be bound by theory, it is believed that a sufficient amount of active agent contained in the oromucosal delivery system is necessary to achieve certain advantageous features of the oromucosal delivery system according to the present invention, such as good in vitro permeation. On the other hand, thick layers may not only provoke an uncomfortable feeling in the oral cavity, but are also difficult to manufacture, and may result in the layer taking too long to dissolve for the desired release profile. In addition, if the amount of active agent is too high, this might lead not only to undesirable storage stability issues such as re-crystallization of the active agent where the active agent is present in dissolved form, but also to potential irritating sensations in the oral cavity due to the drug concentration being too high.

The amount of active agent contained in the oromucosal delivery system can be controlled two-way by adjusting concentration and/or the area weight of the active agent-containing layer. Thus, in certain embodiments of the invention, the active agent-containing layer comprises at least 20 wt-%, at least 25 wt-% or at least 30 wt-% active agent, and/or less than or equal to 60 wt-%, less than or equal to 55 wt-%, or less than or equal to 50 wt % active agent, and/or from 20 to 60 wt-%, from 25 to 55 wt-% or from 30 to 50 wt-% active agent.

In certain embodiments of the invention, the active agent-containing layer comprises at least 4 mg/cm², at least 6 mg/cm² or at least 8 mg/cm² active agent, and/or less than or equal to 15 mg/cm², less than or equal to 13 mg/cm², or less than or equal to 11 mg/cm² active agent, and/or from 4 to 15 mg/cm², from 6 to 13 mg/cm² or from 8 to 11 mg/cm² active agent.

In terms of active amount, the oromucosal delivery system may comprise at least 5 mg, at least 10 mg or at least 15 mg active agent, and/or less than or equal to 80 mg, less than or equal to 70 mg, or less than or equal to 60 mg active agent, and/or from 5 to 80 mg, from 10 to 70 mg or from 15 to 60 mg active agent.

As outlined above, the amounts given here for the active agent are specified in terms of remimazolam in free base form. That is, where an oromucosal delivery system comprises at least 10 mg remimazolam, this corresponds to at least 13.6 mg remimazolam besylate as referred to above.

A correct dissolving behavior of the active agent-containing layer is important for controlling the delivery routes. The faster the disintegration of the oromucosal delivery system, the more dissolution into the saliva will be favored over direct delivery into the mucosa at the adhesion site. In order to achieve a particularly high permeation rate, indirect delivery, which will be able to make use of the whole mucosa for drug delivery, is important. This means that the oromucosal delivery system should disintegrate relatively quickly.

As it is preferred that the active agent-containing layer is able to directly adhere to the mucosa, in certain preferred embodiments of the invention, the active agent-containing layer is mucoadhesive. As will be outlined in further detail below, haptics is an important aspect for oromucosal delivery systems. Thus, in certain embodiments, the active agent-containing layer is in the form of a foam or a flexible monolithic film. Such active agent-containing layers, which are in the form of a foam or which are flexible will provide a more pleasant mouth feel and thus are beneficial in such sense.

In certain embodiments of the present invention, the active agent-containing layer comprises less than or equal to 3 wt-%, less than or equal to 2 wt-%, less than or equal to 1 wt-%, or less than or equal to 0.5 wt-% water.

It has further surprisingly been found that the mucosa permeation rate of the inventive oromucosal delivery systems is favorable at a certain pH. Thus, in some embodiments of the present invention, the oromucosal delivery system is in the form of a film, and, when a sample film of the oromucosal delivery system with a size of 5.75 cm² is dissolved in 5 mL of artificial saliva or of 0.9% NaCl solution, the pH of the resulting solution as measured by a pH electrode ranges from pH 3.0 to pH 3.7.

### Active Agent

In accordance with the invention, the active agent-containing layer comprises remimazolam, a pharmaceutically acceptable salt, or any other form thereof, as active agent, in particular in a therapeutically effective amount.

While in accordance with the present invention, the active agent may be present in the oromucosal delivery system, and in particular in the active agent-containing layer in any form, e.g., in the free base form, in the form of a pharmaceutically acceptable salt, or any mixture thereof, it is preferred that the remimazolam is present in the form of a pharmaceutically acceptable salt.

I.e., in some specific embodiments of the present invention, the oromucosal delivery system contains the active agent in the form of remimazolam besylate or in the form of remimazolam tosylate.

Further, in certain embodiments of the present invention, the active agent in the active agent-containing layer is in dissolved or dispersed form, or is in the form of non-micronized particles.

The active agent in the active agent-containing layer may be (completely) dissolved, or the active agent-containing layer may comprise active agent particles, preferably constituted of the active agent in its free, dissociated form, so that the active agent is present in dispersed form. Needless to say, if the active agent is present in dispersed form, the active agent-containing layer nonetheless may comprise the active agent also in dissolved form, depending on the solubility of the active in the active agent-containing layer (which is e.g. saturated or super-saturated).

In a preferred embodiment, the active agent is completely dissolved, e.g. at least 90 mol %, preferably at least 95 mol %, more preferably at least 98 mol % or most preferably at least 99 mol % of the active agent in the active agent-containing layer is present in dissolved form. It is also preferred that the active agent-containing layer is free of active agent crystals.

As outlined above, the amount of active agent in the oromucosal delivery system is believed to be important for a good release of the active, and can be e.g. adjusted by the active agent concentration. Thus, in certain embodiments, the concentration of active agent in the active agent-containing layer ranges from 20 to 60 wt-%, from 25 to 55 wt-% or from 30 to 50 wt-% of the active agent-containing layer.

The oromucosal delivery systems according to the present invention advantageously show an improved stability in terms of the active agent content as well as active agent degradation.

Thus, in certain embodiments, the active agent-containing layer contains initially (i.e. shortly after manufacture e.g. within one week) an amount of active agent of at least 95%, preferably of at least 97%, more preferably of at least 98% and even more preferably of at least 99% of the theoretical amount of active agent included in the active agent-containing layer. The theoretical amount of active agent is calculated from the active agent amount used for the coating composition and the (actual) area weight of the coated and dried active agent-containing layer of the tested oromucosal delivery system.

The active agent-containing layer may also contain initially a total amount of remimazolam-related degradation substances less than or equal to 0.4 wt-%, less than or equal to 0.3 wt-%, or of less than or equal to 0.2 wt-%.

In certain other embodiments, the oromucosal delivery systems according to the present invention are stable upon storage, i.e. they may maintain the initial remimazolam content values or present low amounts of degradation products, as follows:

In one of such embodiments, the active agent-containing layer contains, after having been stored at 60° C. for up to 6 weeks or 9 weeks, an amount of remimazolam of at least 95%, preferably of at least 97%, more preferably of at least 98% and even more preferably of at least 99% of the theoretical amount of remimazolam included in the active agent-containing layer.

The active agent-containing layer may also contain, when subjected to a storage stability test and after having been stored at 60° C. for up to 6 weeks, a total amount of remimazolam-related degradation substances of less than or equal to 0.5 wt-% or of less than or equal to 0.4 wt-%.

The method for determining the remimazolam content and the total amount of remimazolam-related degradation substances is preferably conducted by validated HPLC-UV as described in the Examples section.

### Film-Forming Agents

As outlined above, the oromucosal delivery system according to the present invention comprises an active agent-containing layer comprising remimazolam, a pharmaceutically acceptable salt, or any other form thereof, as active agent, and a film-forming agent.

This film-forming agent forms a matrix and provides for sufficient cohesion of the active agent-containing layer as long as it is kept in dry state. According to certain embodiments, the film-forming agent may also provide for sufficient adhesion to the mucosa once wet, i.e. when having been brought in contact with the mucosa. In such embodiments, but also in general, film-forming agent may be selected from mucoadhesive polymers.

The film-forming agent is the primary control over the dissolution/disintegration behavior of the active agent-containing layer. By choosing an appropriate film-forming agent, the adhesion to the mucosa as well as disintegration behavior is appropriately adjusted, e.g. in terms of the disintegration time but also in terms of integrity of the oromucosal delivery system.

Film-forming agents which are suitable as the film-forming agent in accordance with the invention are e.g. selected from the group consisting of polymers such as polyvinylpyrrolidone (commercially available as Kollidon® 30F from BASF), methyl cellulose (commercially available as Methocel® from Colorcon), ethyl cellulose (commercially available as Ethocel® from Colorcon), hydroxyethyl cellulose (commercially available as Natrosol® 250 L from Ashland Industries), hydroxypropyl cellulose (commercially available as Klucel® from Ashland Industries), hydroxypropylmethyl cellulose (also known as hypromellose, commercially available as Pharmacoat® from Shin-Etsu), carboxymethyl cellulose sodium (uncrosslinked sodium salt of carboxymethyl cellulose also referred to as CMC or carmellose, commercially available as Blanose® from Ashland Industries), polyethylene glycol-polyvinyl acetate- and polyvinylcaprolactame-based graft copolymers (commercially available as Soluplus® from BASF), polyvinyl alcohol (commercially available as Emprove® from Merck), polyvinyl alcohol-polyethylene glycol copolymers (commercially available as Kollicoat® IR from BASF), polyvinylpyrrolidone-polyvinylacetate copolymers (also referred to as copovidones and commercially available e.g. as Kollidon® VA64 from BASF), polyethylene oxides, polyethylene glycols, methacrylic acid—methyl methacrylate copolymers (commercially available as Eudragit® L100, Eudragit® L12,5, Eudragit® S100 and Eudragit® S12,5 from Evonik), and methacrylic acid—ethyl methacrylate copolymers (commercially available as Eudragit® L100-55 and Eudragit® L30D55 from Evonik), and natural film-forming agents such as shellac, pectin, gelatine, alginate, pullulan and starch derivatives, and any mixtures thereof. Commercially available mixtures are, e.g., Kollidon® SR (a mixture of 80% of polyvinyl acetate and 19% povidone (Kollidon® 30) in a physical mixture, with approximately 0.8% of sodium lauryl sulphate and about 0.2% of silica used as stabilizers) as well as Kollicoat® protect (a mixture comprising 55-65% Polyvinyl alcohol-polyethylene glycol graft copolymer, 35-45% Polyvinyl alcohol and 0.1-0.3% silicon dioxide as processing aid.

The film-forming agent should be able not only to provide sufficient cohesion to the active agent-containing layer, but preferably provides a film that is not tacky in dry state so that the patient is able to touch and manipulate the active agent-containing layer, e.g. to apply the oromucosal delivery system containing the active agent-containing layer to the oral mucosa, without the same adhering to the fingers. In addition, since the film-forming agent is the primary control over the dissolution behavior of the active agent-containing layer which needs to be neither too fast nor too slow, the film-forming agent is preferably soluble, dispersible or otherwise disintegrable in aqueous media, specifically in saliva, or, simplified, in water. On the other hand, in terms of ease of manufacture and for allowing a water-free process of manufacture, which is advantageous in terms of stability of the active agent, film-forming agents that are soluble in other solvents such as C1-C3 alcohols, e.g., ethanol, are also preferred.

The inventors have surprisingly found that, in view of the above, polymers such as polyvinyl alcohol, polyvinyl alcohol-polyethylene glycol grafted copolymers, polyethylene oxides, polyvinylpyrrolidone, polyvinyl caprolactam-polyvinyl acetate-polyethylene glycol graft co-polymer, polyethylene glycols, hydroxypropylmethyl cellulose or any mixture thereof are preferred for the film-forming agents.

Preferred for the film-forming agent are polyvinyl alcohols and polyvinyl alcohol-polyethylene glycol grafted copolymers, particularly polyvinyl alcohols. Polyvinyl alcohol-polyethylene glycol grafted copolymers are commercially available as Kollicoat® IR from BASF. Kollicoat® IR is particularly preferred and is a polymer consisting of 75% polyvinyl alcohol units and 25% polyethylene glycol units, containing further approx. 0.3% colloidal silica to improve flow properties, with a MW of 45,000 AMU. Polyvinyl alcohols are commercially available from Kuraray under the brand name Mowiol and from Merck under the brand name Parteck® MXP and Emprove® and are provided in several grades, differing in the degree of hydrolysis and in the molecular weight. In certain preferred embodiments, the film-forming agent is a polyvinyl alcohol having a molecular weight in the range from 10,000 to 250,000, or a mixture of two or more polyvinyl alcohols, each having a molecular weight in the range from 10,000 to 250,000. The molecular weight is determined as mean weights of the molar masses Mw by gel permeation chromatography (GPC) combined with static light scattering (absolute method) on re-acetylized specimens.

The Mowiol partially hydrolyzed grades differ from each other by molecular weight MW and are as follows:
the PVA 3-83 has a MW of about 14,000
the PVA 4-88 has a MW of about 31,000
the PVA 5-88 has a MW of about 37,000
the PVA 8-88 has a MW of about 67,000
the PVA 18-88 has a MW of about 130,000
the PVA 23-88 has a MW of about 150,000
the PVA 26-88 has a MW of about 160,000
the PVA 40-88 has a MW of about 205,000.
   Whereas the Mowiol fully hydrolyzed grades differ from each other by molecular weight MW and are as follows:
the PVA 3-98 has a MW of about 16,000
the PVA 4-98 has a MW of about 27,000
the PVA 6-98 has a MW of about 47,000
the PVA 10-98 has a MW of about 67,000
the PVA 20-98 has a MW of about 130,000
the PVA 56-98 has a MW of about 150,000
the PVA 28-99 has a MW of about 160,000
   The Parteck® MXP 4-88 grade PVA has a MW of 32,000, and the Parteck® MXP 3-82 grade PVA has a MW of 47,000. In these grade names, the first number provides the apparent viscosity in mPa·s of a 4% aqueous solution at 20° C. and the second number provides the hydrolysis grade in %. So, Parteck® MXP 3-82 shows a viscosity of 3 mPa·s and a hydrolysis grade of 82%, while Parteck® MXP 4-88 shows a viscosity of 4 mPa·s and a hydrolysis grade of 88%.

In order to be able to provide sufficient cohesion to the active agent-containing layer, a certain amount of the film-forming agent should be included. Thus, in certain preferred embodiments, the active agent-containing layer comprises at least 10 wt-%, at least 13 wt-% or at least 15 wt-% of the film-forming agent, less than or equal to 75 wt-%, less than or equal to 50 wt-%, or less than or equal to 30 wt % of the film-forming agent, and/or from 10 to 75 wt-%, from 13 to 50 wt-% or from 15 to 30 wt-% of the film-forming agent.

However, the amount of film-forming agent should be adapted depending on the presence of further excipients in the formulation. As will be outlined in the next chapter, the above numbers are taking into account that a certain amount of plasticizer may be present.

In other embodiments, the active agent-containing layer does not comprise a plasticizer and comprises at least 50 wt-%, at least 60 wt-% or at least 65 wt-% of the film-forming agent, less than or equal to 85 wt-%, less than or equal to 75 wt-%, or less than or equal to 70 wt-% of the film-forming agent, and/or from 50 to 85 wt-%, from 60 to 75 wt-% or from 65 to 70 wt-% of the film-forming agent. The active agent-containing layer may also substantially consist of the active agent and the film-forming agent.

A film-forming agent as disclosed above may be present as the film-forming agent in the active agent-containing layer, but may also be contained in any further (optional) layer or an optional overlay.

### Plasticizer

The active agent-containing layer of the oromucosal delivery system according to the present invention comprises remimazolam, a pharmaceutically acceptable salt, or any other form thereof, as active agent, and a film-forming agent. Depending on factors such as the type and amount of the film-forming agent, the form and amount of the active agent but also further constituents of the formulation or the manufacturing process, it may be beneficial to incorporate a plasticizer. A plasticizer will provide for more flexibility of the active agent-containing layer and will reduce the risk of the layer getting brittle, also over time. Plasticizers may also improve the haptics of the oromucosal delivery system upon administration to the oral mucosa, i.e., provide a good feeling in the mouth.

Therefore, in some embodiments of the present invention, the active agent-containing layer may further comprise a plasticizer.

In other words, in some specific embodiments, the active agent-containing layer comprises:
i) remimazolam, a pharmaceutically acceptable salt, or any other form thereof, as active agent,
ii) a film-forming agent, and
iii) a plasticizer.

The plasticizer selected from the group consisting of linear or branched, saturated or unsaturated alcohols having 6 to 20 carbon atoms, triglycerides, polyethylene glycol or polyvinyl alcohol-polyethylene glycol grafted copolymers. Particularly preferred are polyethylene glycols and polyvinyl alcohol-polyethylene glycol grafted copolymers. In a further preferred embodiment, a polyvinyl alcohol-polyethylene glycol grafted copolymer is used as plasticizer and the film-forming agent is a polyvinyl alcohol, preferably with a molecular weight of 31,000 or 205,000 (grades 4-88 and 40-88) or any mixture thereof.

In certain embodiments of the present invention, the active agent-containing layer comprises at least 5 wt-%, at least 15 wt-% or at least 20 wt-% of the plasticizer, less than or equal to 50 wt-%, less than or equal to 40 wt-%, or less than or equal to 35 wt % of the plasticizer, and/or from 5 to 50 wt-%, from 15 to 40 wt-% or from 20 to 35 wt-% of the plasticizer.

In order to provide for a good balance between the amount of film-forming agent and plasticizer, which on the one hand provide for the matrix of the active agent-containing layer, and the amount of active agent, it is useful to keep the total amount of film-forming agent as well as plasticizer in a certain range or to provide the two components in a specific ratio.

Thus, in some specific embodiments of the present invention, the active agent-containing layer comprises a total amount of film-forming agent and plasticizer, wherein said total amount is at least 30 wt-%, at least 35 wt-% or at least 40 wt-% of the active agent-containing layer, less than or equal to 80 wt-%, less than or equal to 60 wt-%, or less than or equal to 45 wt % of the active agent-containing layer, and/or from 30 to 80 wt-%, from 35 to 60 wt-% or from 40 to 45 wt-% of the active agent-containing layer. In also specific embodiments, the active agent-containing layer comprises a plasticizer, wherein the film-forming agent is a polyvinyl alcohol, and wherein the ratio of polyvinyl alcohol to the plasticizer is at least 20:80, or is less than or equal to 50:50, or is from 20:80 to 50:50, or is about 25:75 or about 40:60.

In certain embodiments of the present invention, the active agent-containing layer does not comprise a plasticizer and comprises at least 50 wt-%, at least 60 wt-% or at least 65 wt-% of the film-forming agent, less than or equal to 85 wt-%, less than or equal to 75 wt-%, or less than or equal to 70 wt-% of the film-forming agent, and/or from 50 to 85 wt-%, from 60 to 75 wt-% or from 65 to 70 wt-% of the film-forming agent, and/or the active agent-containing layer substantially consists of (i) the active agent and (ii) the film-forming agent.

### Further Excipients

The active agent-containing layer of the oromucosal delivery system according to the invention may comprise further excipients common in the art such as fatty acids, sweeteners, flavoring agents, colorants, permeation enhancers, solubilizers, plasticizers, humectants, disintegrants, emulsifiers, antioxidants, stabilizers, buffer reagents and further film-forming agents.

In certain embodiments, the active agent-containing layer further comprises one or more excipients selected from the group consisting of sweeteners, flavoring agents, antioxidants and pH adjusting agents. As outlined above, the active agent is preferably present in form of the besylate or tosylate acid-addition salt, which may provide for the desired pH value. The presence of pH adjusting agents thus does not seem to be always necessary, i.e., in certain embodiments, the active agent-containing layer does not comprise a pH adjusting agent.

The excipients may be present in the active agent-containing layer in an amount of from 0.001 to 15 wt-% of the active agent-containing layer per excipient. In a certain embodiment, the total amount of all excipients is from 0.001 to 25 wt-% of the active agent-containing layer. Hereinafter, where a range for an amount of a specific additive is given, such a range refers to the amount per individual additive.

It should be noted that in pharmaceutical formulations, the formulation components are categorized according to their physicochemical and physiological properties, and in accordance with their function. This means in particular that a substance or a compound falling into one category is not excluded from falling into another category of formulation component. In such a case, for the purpose of calculations in a patent claim (e.g. for determining weight percentages, ratios etc.), such a substance or compound may preferably be allocated to the fitting category that is mentioned first in the respective claim. E.g. a certain polymer such as the polyvinyl alcohol-polyethylene glycol grafted copolymer can be a plasticizer, but also a film-forming agent. Some substances may e.g. be a typical softener but at the same time act as a permeation enhancer. The skilled person is able to determine based on his general knowledge in which category or categories of formulation component a certain substance or compound belongs to. In the following, details on the excipients and additives are provided which are, however, not to be understood as being exclusive. Other substances not explicitly listed in the present description may be as well used in accordance with the present invention, and substances and/or compounds explicitly listed for one category of formulation component are not excluded from being used as another formulation component in the sense of the present invention.

Substances that are able to mask or modify the taste, or which might otherwise alleviate any potential unpleasant effect of remimazolam, are particularly preferred as excipient.

Thus, in certain preferred embodiments, the active agent-containing layer further comprises one or more excipients selected from the group consisting of sweeteners and flavoring agents.

In certain preferred embodiments, the active agent-containing layer comprises one or more natural or artificial sweeteners, which may be selected from the group consisting of saccharose, glucose, fructose, sorbitol, mannitol, isomalt, maltitol, lactitol, xylitol, erythritol, sucralose, acesulfame potassium, N—[N-[3-(3-Hydroxy-4-methoxyphenyl)propyl]-α-L-aspartyl]-L-phenylalanin-1-methylester (advantame), N—[N-(3,3-Dimethylbutyl)-L-α-aspartyl]-L-phenylalanine 1-methyl ester (neotame), aspartame, cyclamate, neohesperidine, neotame, steviol glycosides, thaumatine and saccharin sodium. Preferably, the sweeteners are selected from the group consisting of sucralose, acesulfame potassium, advantame, N—[N-(3,3-Dimethylbutyl)-L-α-aspartyl]-L-phenylalanine 1-methyl ester (neotame), aspartame, thaumatine, and particularly preferably, the sweetener is N—[N-(3,3-Dimethylbutyl)-L-α-aspartyl]-L-phenylalanine 1-methyl ester (neotame) or N—[N-[3-(3-Hydroxy-4-methoxyphenyl)propyl]-α-L-aspartyl]-L-phenylalanin-1-methylester (advantame) or a mixture thereof. In such embodiments, i.e. wherein the active agent-containing layer comprises one or more natural or artificial sweeteners, the active agent-containing layer may comprise at least 0.05 wt-% or at least 0.5 wt-% of a sweetener, less than or equal to 2 wt-% or less than or equal to 1 wt-% of a sweetener, and/or from 0.05 wt-% to 2 wt-% or from 0.5 to 1 wt-% of a sweetener.

In also preferred embodiments, the active agent-containing layer comprises one or more natural or artificial flavoring agents selected from the group consisting of vanillin, methyl salicylate, menthol, manzanate, diacetyl, acetylpropionyl, acetoin, isoamyl acetate, benzaldehyde, cinnamaldehyde, ethyl propionate, methyl anthranilate, limonene, ethyl decadienoate, allyl hexanoate, ethyl maltol, 2,4-dithiapentane, ethylvanillin and eucalyptol as well as flavoring compositions such as peppermint flavor and MANE flavors such as MANE Orange flavor or MANE Tutti Frutti Flavor. Flavoring compositions such as MANE Orange flavor, which is a combination of Linalool, Alpha Pinene, Citral, Delta 3 Carene, Beta Pinene and Myrcene, and MANE Tutti Frutti flavor which is a combination of Geranyle Acetate, Vanillin, Limonene and Allyl hexanoate, are preferred. In such embodiments, i.e. wherein the active agent-containing layer comprises one or more flavoring agents, the active agent-containing layer may comprise at least 0.05 wt-% or at least 0.5 wt-% of a flavoring agent, less than or equal to 5 wt-% or less than or equal to 2 wt-% of a flavoring agent, and/or from 0.05 wt-% to 5 wt-% or from 0.5 to 2 wt-% of a flavoring agent.

Suitable flavoring agents are also commercially available from the company Mane, and any of those, which are identified by tonalities such as apple, caramel, chocolate, lemon, mint, etc. can be used as a flavoring agent in the present invention.

The active agent-containing layer according to the invention may comprise a pH adjusting agent, as indicated above. The pH adjusting agent may be, e.g., selected from mono- and polytropic acids, mono-, di- and triacidic bases, buffer solutions with mixtures of a weak acid and its conjugate base, amine derivatives, inorganic alkali derivatives, polymers with basic and acidic functionality, respectively. The active agent-containing layer according to the invention may also comprise an antioxidant. Suitable antioxidants are, e.g., ascorbic acid, α-tocopherol, ascorbyl palmitate and sodium meta bisulfite.

### Pharmaceutical Product

As outlined above, according to one aspect, the present invention relates to a pharmaceutical product comprising a packaging and one or more unit doses of the oromucosal delivery system.

In certain embodiments, the packaging is in the form of a pouch. The pouch may be made of a multi-layer film material comprising an outward-facing paper layer, an intermediate polyethylene layer and an inward-facing aluminum layer. To protect the unit doses of oromucosal delivery system contained, e.g., from humidity or oxygen, the pouch may be sealed with a sealant. In certain embodiments, the sealant is selected from the group consisting of an ethylene copolymer (modified ionomer, commercially available, e.g., as Surlyn®), or a polyethylene terephthalate copolymer and a cyclic olefin copolymer.

In certain embodiments, as a further protective measure against humidity and associated degradation of the active agent, the pouch is nitrogen-filled. The pouch may also comprise one or more desiccant(s), but does not necessarily comprise a desiccant. In certain embodiments, the desiccant comprises silica gel, molecular sieves 4 Å and/or zeolite molecular sieves 4 Å as a drying agent, and/or is in the form of an adhesive film. The adhesive film can be attached on the inward-facing side of the pouch. Such an adhesive dessicant is commercially available as DesiMax.

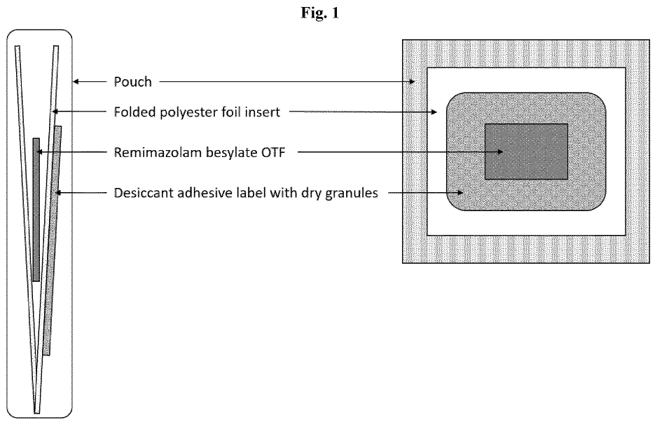

In further embodiments, the pharmaceutical product comprises a folded polyethylene terephthalate foil within the primary packaging, wherein the one or more unit dose(s) of the oromucosal delivery system are enclosed by the folded polyethylene terephthalate foil which is folded around and protects the unit dose(s) from further contact with the packaging. In such a case, the pouch may comprise one or more desiccant(s) in the form of an adhesive film attached on the outward-facing side of the folded polyethylene terephthalate foil so that contact with the oromucosal delivery system is avoided. Such a configuration is shown in FIG. 1. In other embodiments, the pharmaceutical product does not comprise such a folded polyethylene terephthalate foil.

### Method of Producing Sedation and Other Methods

In accordance with a specific aspect of the present invention, the oromucosal delivery system according to the invention is for use in producing sedation, hypnosis, anxiolysis, muscle relaxation, the treatment of convulsions or the induction of amnesia for perioperative events. In accordance with another aspect, the present invention is related to a method of producing sedation, hypnosis, anxiolysis, muscle relaxation, the treatment of convulsions or the induction of amnesia for perioperative events, wherein the oromucosal delivery system is administered to a subject, and preferably a human patient. In yet another aspect, the present invention relates to the use of the oromucosal delivery system in the preparation of a medicament for producing sedation, hypnosis, anxiolysis, muscle relaxation, the treatment of convulsions or the induction of amnesia for perioperative events.

Remimazolam is, as explained above, a very promising ultra-short acting sedative that has been approved both for procedural sedation as well as for general anesthesia.

Thus, in certain embodiments, sedation as referred to in the different aspects above is procedural sedation such as sedation for dental procedures or sedation for diagnostic procedures, preoperative sedation, and/or conscious sedation. The sedation may be induced before and/or during various procedures such as endoscopy, colonoscopy, or other diagnostic or operative procedures. As used herein, “procedural sedation” includes, but is not limited to, sedation for the performance of an endoscopy (preferably upper GI endoscopy or colonoscopy), a dental procedure, a diagnostic procedure, imaging or a short unpleasant procedure (such as e.g. changing of wound dressings or manipulations of a catheter, e.g. the removal of a central catheter).

In terms of the sedative effect, in certain embodiments, mild sedation, moderate sedation, deep sedation or general anesthesia is reached. In certain embodiments, the sedation or general anesthesia reached has a duration of 5 to 30 minutes, 8 to 20 minutes or 10 to 15 minutes.

To achieve sedation, in certain embodiments, the oromucosal delivery system is administered by applying the active agent-containing layer to the mucosa, and in particular to the buccal, sublingual, gingival or palatal mucosa, of the oral cavity of a human patient and maintained on the mucosa until dissolved.

As outlined above, transmucosal delivery avoids the first-pass effect and thus, the oromucosal delivery system according to the invention has a lower risk of any unintended effects such as those due to hepatic metabolism, and is favorable in terms of patient acceptance when compared to IV administration. Thus, there is no limitation with respect to the patient group. The subjects may be human patients with or without hepatic impairment, including also elderly as well as children.

### Process of Manufacture

The invention further relates to a process of manufacture of an active agent-containing layer for use in an oromucosal delivery system and a corresponding active agent-containing layer and a corresponding oromucosal delivery system.

In accordance with the invention, the process of manufacture of an active agent-containing layer comprises the steps of:
i. Combining at least (i) remimazolam, a pharmaceutically acceptable salt, or any other form thereof, as active agent, and (ii) a film-forming agent to obtain a mixture; and
ii. Forming the active agent-containing layer.

In such a process, suitable film-forming agents are the same as those mentioned previously.

Step ii. of forming the active agent-containing layer can be conducted by any known method. In certain embodiments, the process is a hot-melt process, a coating process or a foam forming process.

The hot-melt process may be a hot-melt extrusion process or a vacuum compression molding process, wherein:
the hot-melt extrusion process comprises step ii) of:
   a. introducing the mixture comprising the active agent and the film-forming agent with or without further excipients into an extruder;
   b. heating said mixture to at least the softening temperature of said mixture; and
   c. extruding the heated mixture comprising the film-forming agent and the active agent in the form of a film to obtain the active agent-containing layer,
   while
the vacuum compression molding process comprises step ii) of:
   a. introducing the mixture comprising the active agent and a film-forming agent with or without excipients into a sample chamber; and
   b. compacting said mixture while applying vacuum and heating said mixture to at least the softening temperature of said mixture to obtain the active agent-containing layer.

The coating process on the other hand may comprise the steps of:
i. dispersing or dissolving the active agent in a solution of the film-forming agent with or without further excipients to obtain a coating composition, and
ii. a. coating the coating composition on a coating substrate; and
   b. drying the laminate segments in a drying oven to obtain the active agent-containing layer in the form of a flexible monolithic film.

Finally, the foam forming process may comprise the steps of:
i. dispersing or dissolving the active agent in an aqueous solution of the film-forming agent with or without further excipients to obtain a coating composition, and
ii. a. foaming the coating composition to obtain a foam coating composition;
   b. coating the foam coating composition on a coating substrate; and
   c. drying the laminate segments in a drying oven to obtain the active agent-containing layer in the form of a foam.

In such an embodiment, foaming can be conducted by stirring while introducing nitrogen gas into the composition. In such an embodiment, the stirring may be in particular at high speed, and/or using a foaming device comprising a foaming head with a dispersion unit.

According to a further aspect, the invention relates to a process of manufacture of an oromucosal delivery system comprising an active agent-containing layer comprising (i) remimazolam, a pharmaceutically acceptable salt, or any other form thereof, as active agent, and (ii) a film-forming agent comprising the steps of:
i. Combining at least the active agent and a film-forming agent to obtain a mixture; and
ii. Forming the active agent-containing layer.

According to a further aspect, the invention relates to an oromucosal delivery system obtainable by such a process.

According to certain embodiments, one or more unit doses of the oromucosal delivery system can be packaged in a primary packaging to provide a pharmaceutical product. In such an embodiment, the primary packaging may be in the form of a pouch as described further above. The step of packaging can be conducted under nitrogen atmosphere to obtain nitrogen-filled pouches which protect against environmental humidity.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability.

### Examples 1A-1H

### Preparation of the Coating Composition (Active Agent-Containing Layer) and Coating of the Coating Composition

The formulations for Examples 1a-1h are summarized in Tables 1-6. In these tables and in the following, “Amt [g]” refers to the amount in gram. The active agent remimazolam besylate was ground by means of a mortar.

For Example 1a, a beaker was loaded with remimazolam (in the following also denoted as RMZ) besylate. Then aqua purificata was added and the mixture was stirred for 1 min at 1000 rpm. Then PEG300 was added and it was again stirred for 1 min at 1000 rpm. Kollicoat® Protect was added while stirring at 1000-2000 rpm. After the last addition, stirring was continued for 5 min at 2000 rpm.

The resulting active agent-containing foam coating composition was coated on polyethylene terephthalate (100 μm thickness) and was dried for 45 min at 70° C. The coating thickness gave an area weight of 232 g/m².

TABLE 1
Example 1a - Foam (API dispersed)

| | |
|---|---|
| Solids [%] | Amt [g] |

Ingredient (Trade Name)

| | | |
|---|---|---|
| RMZ besylate | 22.6 | 2.38 |
| PEG 300 | 5.1 | 0.54 |
| Kollicoat protect | 72.2 | 7.59 |

Solvent

| | | |
|---|---|---|
| Aqua purificata | n.a. | 19.62 |
| Solids content | 100 | 10.51 |

Film characteristics

| | |
|---|---|
| Area weight [g/m²] | 232 |
| RMZ besylate -content [mg/system] | 34.3 |
| system size [cm²] | 6 |

For Example 1b, a beaker was loaded with remimazolam besylate and PEG 300 and 199.61 g aqua purificata were added. The beaker was swirled and then a 26.2 g of a solution of 14.48 g Methocel E3LV and 8.07 g Methocel E50LV in 127.55 g aqua purificata were added. The mixture was stirred for 10 min at 1000 rpm, followed by stirring for 5 min at 2000 rpm and by stirring for 5 min at 100 rpm.

The resulting active agent-containing coating composition was coated on polyethylene terephthalate (100 μm thickness) and was dried for 45 min at 70° C. The coating thickness gave an area weight of 103 g/m².

TABLE 2
Example 1b - Film (API dispersed)

| | |
|---|---|
| Solids [%] | Amt [g] |

Ingredient (Trade Name)

| | | |
|---|---|---|
| RMZ besylate | 29.8 | 1.82 |
| PEG 300 | 5.6 | 0.34 |
| Methocel E3LV | 41.5 | 2.53 |
| Methocel E50LV | 23.1 | 1.41 |

Solvent

| | | |
|---|---|---|
| Aqua purificata | n.a. | 221.9 |
| Solids content | 100 | 6.1 |

Film characteristics

| | |
|---|---|
| Area weight [g/m²] | 107 |
| RMZ besylate-content [mg/system] | 30.8 |
| system size [cm²] | 9 |

For Example 1c, remimazolam besylate was loaded in a beaker and PEG 300 as well as 87.7 g of a solution of 700 g PVA 4-88 in 1300.1 g aqua purificata was added. The mixture was stirred until remimazolam besylate was dispersed in the mixture and then a foam was formed.

The resulting active agent-containing foam coating composition was coated on Polyslik 111/80 (one side siliconized) and dried for 15 min at 70° C. The coating thickness gave an area weight of 181 g/m².

TABLE 3
Example 1c - Foam (API dispersed)

| | |
|---|---|
| Solids [%] | Amt [g] |

Ingredient (Trade Name)

| | | |
|---|---|---|
| RMZ besylate | 22.7 | 9.75 |
| PEG 300 | 6.00 | 2.59 |
| PVA 4-88 | 71.3 | 30.69 |

Solvent

| | | |
|---|---|---|
| Aqua purificata | n.a. | 56.99 |
| Solids content | 100 | 43.03 |

Film characteristics

| | |
|---|---|
| Area weight [g/m²] | 181 |
| RMZ besylate -content [mg/system] | 28.7 |
| system size [cm²] | 7 |

For Example 1d, a heating jacket was preheated to 150° C. A beaker was loaded with Polyox N10 and the beaker was placed in the preheated heating jacket. PolyoxN10 was stirred for 45 min at 57 rpm, then for 65 min at 150 min and then for 130 min at 250 rpm. The temperature for the PolyoxN10 was 136° C. Remimazolam besylate was added under stirring at 300 rpm. Stirring was continued for 15 min at 520 rpm. The temperature was 135° C. at this point.

The resulting active agent-containing coating composition was coated on polyethylene terephthalate (100 μm thickness) using a hot-melt coater with both top and bottom roller being heated to 150° C. and with a gap width of 405 μm. The coating thickness gave an area weight of 355 g/m².

TABLE 4
Example 1d - Hotmelt (API dispersed)

| | |
|---|---|
| Solids [%] | Amt [g] |

Ingredient (Trade Name)

| | | |
|---|---|---|
| RMZ besylate | 22.7 | 11.34 |
| Polyox N10 | 77.3 | 38.67 |
| Solids content | 100 | 50.01 |

Film characteristics

| | |
|---|---|
| Area weight [g/m²] | 355 |
| RMZ besylate -content [mg/system] | 25.3 |
| system size [cm²] | 3.14 |

For Examples 1e and 1g, a beaker was loaded with aqua purificata and the film-forming agent (Kollidon VA64 or Soluplus) was added under stirring at 1500 rpm. Stirring was continued for 1.25 h at 250 rpm followed by swelling overnight. On the next day, a beaker was loaded with remimazolam besylate. PEG 300 was added followed by mixing by hand. Then, the swollen mixture of the film-forming agent in aqua purificata was added followed by stirring of the newly formed mixture for 45 min at 250 rpm.

The active agent-containing coating composition was coated on Polyslik (one side siliconized) and dried for 25 min at 70° C. The coating thickness gave an area weight of 244 g/m² (1e) or 123 g/m² (1g).

For Examples 1f and 1h, a beaker was loaded with remimazolam besylate and methanol was added until the remimazolam besylate was dissolved. Then the film-forming agent (Kollidon VA64 or Soluplus) was added under stirring followed by the addition of PEG 300 to the mixture.

The active agent-containing coating composition was coated on Polyslik 111/80 (one side siliconized) and dried for 30 min at 70° C. The coating thickness gave an area weight of 63 g/m².

TABLE 5
Example 1e & 1f - Film (API dispersed)

| Example | | | |
|---|---|---|---|
| 1e | | 1f | |
| Solids | Amt | Solids | Amt |
| [%] | [g] | [%] | [g] |

Ingredient (Trade Name)

| | | | | |
|---|---|---|---|---|
| RMZ besylate | 30.5 | 2.18 | 30.0 | 2.05 |
| PEG 300 | 15.7 | 1.12 | 16.3 | 1.11 |
| Kollidon VA64 | 53.8 | 3.85 | 53.7 | 3.67 |

Solvent

| | | | | |
|---|---|---|---|---|
| Aqua purificata | — | 2.91 | — | — |
| Methanol | — | — | n.a. | 10.37 |
| Solids content | 100 | 7.15 | 100 | 6.83 |

Film characteristics

| | | |
|---|---|---|
| Area weight [g/m²] | 244 | 63 |
| RMZ besylate-content [mg/system] | 24.5 | 38.4 |
| system size [cm²] | 3.5 | 15 |

TABLE 6
Examples 1g & 1h - Film (API dispersed)

| Example | | | |
|---|---|---|---|
| 1g | | 1h | |
| Solids | Amt | Solids | Amt |
| [%] | [g] | [%] | [g] |

Ingredients (Trade Name)

| | | | | |
|---|---|---|---|---|
| RMZ besylate | 30.2 | 2.96 | 29.7 | 2.02 |
| PEG 300 | 16.1 | 1.58 | 16.3 | 1.11 |
| Soluplus | 53.7 | 5.26 | 54.0 | 3..67 |

Solvents

| | | | | |
|---|---|---|---|---|
| Aqua purificata | | 4.47 | — | — |
| Methanol | — | — | n.a. | 9.85 |
| Solids content | 100 | 9.8 | 100 | 6.8 |

Film characteristics

| | | |
|---|---|---|
| Area weight [g/m²] | 123 | 87 |
| RMZ besylate-content [mg/system] | 28.7 | 22.7 |
| system size [cm²] | 7.5 | 10 |

### Preparation of the Oromucosal Delivery System (Concerning all Examples)

Individual oromucosal delivery systems were then punched out from the active agent-containing layer. The oromucosal delivery systems were then sealed into water steam proof Surlyn® pouches. For Example 1D, the oromucosal delivery system was sealed into a Surlyn® pouch together with two water absorber patches (DesiMax).

### Stability Study to Evaluate the Stability of the Remimazolam Besylate in the Different Formulations

The prepared oromucosal delivery systems according to Examples 1a-1h were sealed in Surlyn® packaging material and stored at 25° C./60% RH and 40° C./75% RH conditions for 2, 4 and 8 weeks. Samples were analyzed in order to obtain rapid stability assessment of potential API-excipient-solvent interactions which might lead to any instability resulting in the formation of new impurities and/or degradation products (e.g. via hydrolysis). Tests for remimazolam content and degradation products were performed after 2 weeks, 4 weeks and 8 weeks of storage.

In defined intervals shown in Table 7, samples were drawn and tested for assay and Remimazolam degradation products. Therefore, the 20.0 mL of diluent (H₂O/ACN 3:1 (v/v)) were added to the respective sample and stirred for approx. 45 min. Subsequently, an aliquot of the resulting sample solution was centrifuged at 10.000 rpm for 10 min (at approx. 5° C.) and analyzed by a validated HPLC-UV method. Drug-free matrix samples served as references to enable assignment of potential interferences coming from the excipients used. The results are shown in Table 8.

TABLE 7
Sampling and testing plan for each API
containing Oromucodal delivery system

Storage conditions (° C./% RH)

| | | |
|---|---|---|
| Storage | 25/60 | 40/75 |

| | |
|---|---|
| Initial | n = 3 |

| | | |
|---|---|---|
| 2 weeks | n = 3¹ | n = 3 |
| 4 weeks | n = 3¹ | n = 3 |
| 8 weels | n = 3¹ | n = 3 |

¹Only analyzed if 40° C. samples show degradation.

TABLE 8

| | Impurities (%) | | | | | | |
|---|---|---|---|---|---|---|---|
| | a = CNS7054 (hydrolysis product) | | | | | | |
| | b = CNS7084 | | | | | | |
| | c = unknown impurities | | | | | | |
| | Initial | 2 weeks | | 4 weeks | | 8 weeks | |
| Example | 25° C. | 25° C. | 40° C. | 25° C. | 40° C. | 25° C. | 40° C. |
| 1a | 0.3 a | n.t | 0.4 a | 0.6 a | 0.4 a | 0.5 a | 0.6 a |
| | 0.3 b | | 0.3 b | 0.5 b | 0.3 b | 0.4 b | 0.5 b |
| | 0.2 c | | 1.1 c | 0.9 c | 1.9 c | 1.3 c | 2.0 c |
| 1b | 1.7 a | n.t. | 7.1 a | 4.2 a | n.t. | | |
| | 0.5 b | | 6.0 b | 1.7 b | | | |
| | 0.2 c | | 1.4 c | 0.6 c | | | |
| 1c | 0.1a | n.t. | 0.6 a | 0.5 a | 0.6 a | 0.4 a | 0.7 a |
| | 0.2 b | | 0.9 b | 0.3 c | 0.9 b | 0.4 b | 0.9 b |
| | | | 0.6 c | 0.3 c | 0.8 c | 0.5 c | 1.0 c |
| 1d | <0.1a | n.t. | 0.1 a | 0.2 a | 0.1 a | <0.1 a | 0.3 a |
| | | | <0.1 b | | <0.1 b | | <0.1 b |
| | | | 0.2 c | 0.1 c | 0.7 c | <0.1 c | 1.6 c |
| 1e | 0.8 a | n.t. | 2.1 a | 1.7 a | 2.7 a | n.t. | |
| | | | <0.1 b | <0.1 b | 0.2 b | | |
| | | | 0.4 c | 0.2 c | 0.7 c | | |
| 1f | 3 a | n.t. | | | | | |
| 1g | 1.0 a | n.t. | 1.8 a | 1.9 a | 2.4 a | n.t. | |
| | 0.1 b | | <0.1 b | <0.1 b | <0.1 b | | |
| | | | 0.2 c | 0.2 c | 0.3 c | | |
| 1h | 1.7 a | n.t. | 2.9 a | 5.5 a | 3.7 a | n.t. | |
| | 0.6 b | | 0.4 b | 1.1 b | 0.3 b | | |
| | | | 0.2 c | 0.4 c | 0.3 c | | |

n.t. not further investigated due to high degradation

In all formulations, the degradation products CNS7054 (hydrolysis) and CNS7084 were measured in varying amounts. Moreover, unknown impurities were detected in all formulations in different amounts.

For PVA 4-88-based foam (Example 1c) and Polyox™ N10-based hot melt (Example 1d) formulations, the amount of degradation products was in an acceptable range, but for both formulations, unknown impurities were detected. For all other approaches, high degradation (hydrolysis in particular) and unknown impurities were detected.

In Vivo Studies with Example 1c and 1d Using Goettingen Minipigs

In order to assess the pharmacokinetic profile and effect of buccally administered oromucosal delivery systems containing remimazolam besylate, in vivo experiments using Goettingen minipigs (female, about 3 months old, body weight was 7 kg at the start of the study) were conducted. Three minipigs were used. A single oromucosal delivery system of Examples 1c (foam) and 1d (hotmelt), prepared as described above, with a nominal amount of 20 mg remimazolam (formulated as 27.2 mg nominal remimazolam besylate), was used per animal (with the exception of animal No 3 in Phase 1, where only half of an oromucosal delivery system of each type was placed in each side). The oromucosal delivery system foam was cut into two pieces to fit on the buccal mucosa. The groups, dose levels and animal numbers are summarized in Table 9.

TABLE 9

| | Administration route, test item and dose | | |
|---|---|---|---|
| Phase | Animal No 1 | Animal No 2 | Animal No 3 |
| 1 | system -placebo | system -placebo | ½ system -placebo |
| | foam | hotmelt | foam (right side) and |
| | | | ½ system -placebo |
| | | | hotmelt (left-side) |
| 2 | IV, RMZ, 10 mg | system -hotmelt, | system -foam, RMZ, |
| | (2 mL of 5 mg/mL) | RMZ, 20 mg | 20 mg |
| 3 | system -hotmelt, | system -foam, | IV, RMZ, 10 mg |
| | RMZ, 20 mg | RMZ, 20 mg | (2 mL of 5 mg/mL) |
| 4 | system -foam, | IV, RMZ, 10 mg | system -hotmelt, |
| | RMZ, 20 mg | (2 mL of 5 mg/mL) | RMZ, 20 mg |

The formulations of the placebo systems corresponding to Examples 1c (foam) and 1d (hotmelt) are summarized in Table 10 below. The coating compositions were prepared as described for Examples 1c and 1d above.

TABLE 10

| | Formulation | | Composition | |
|---|---|---|---|---|
| No. | type | Function | (% w/w) | Solvent |
| 1c | Placebo, | Technical | 93.7 PVA 4-88 | Water |
| placebo | foam | placebo sample | 6.3 PEG 300 | |
| 1d | Placebo, | Technical | 100 Polyox ™ | n.a. |
| placebo | hot-melt | placebo sample | WSR N10 | |

The dose was given by buccal administration (oromucosal delivery system; hotmelt and foam) or IV administration (10 mg remimazolam formulated as remimazolam besylate according to the treatment schedule shown in table 9. No less than 48 hours were allowed between dosing occasions to allow for proper wash-out. The treatment with the oromucosal delivery systems was performed as follows:
1. The animals were anaesthetized by mask with isoflurane to obtain sufficient depth of anesthesia to facilitate the placement of the oromucosal delivery system without stressing the animals. The length and concentration of isoflurane anesthesia was similar in all animals.
2. The oromucosal delivery system was placed on the buccal mucosa. A small amount of saline was applied to the mucosa to facilitate the dissolution process on a single occasion in Phase 1.
3. The mouth of the minipigs was closed for 1-2 minutes and then opened to check for completely dissolved oromucosal delivery system. If the oromucosal delivery system was not dissolved further checks were performed with intervals of 1-2 minutes, as required.
4. The isoflurane treatment was stopped as soon as the oromucosal delivery system was dissolved.

In order to evaluate the bioavailability, the animals also received a single intravenous (IV) dose. The IV dose volume was administered as a bolus over a period of approximately 1 minute.

The RMZ IV dose formulation was prepared in glass containers. To prepare said formulation of nominal 5 mg/mL (stock solution), a drug product vial (50 mg of RZM powder) was reconstituted with 10 mL of 0.9% w/v sodium chloride solution. The reconstituted solutions of RMZ were clear and colorless to slightly yellow. The reconstituted RMZ drug product was used within 24 hours.

No local reactions were observed at the administration sites at 90 minutes post dosing.

Reflexes and depth of sedation were monitored and recorded every 5 minutes from end of isoflurane use until the animals were fully awake. In Phase 1, the time from induction to full recovery from isoflurane sedation was recorded. The depth of sedation was scored as follows in Table 11.

TABLE 11

| Sedation/ | | |
|---|---|---|
| anaesthesia | Level of sedation | |
| score | or anaesthesia | Criteria |
| 0 | No sedation | Normal behaviour |
| 1 | Recovering from | Able to stand but ataxic |
| | sedation | |
| 2 | Mild sedation | Sternal recumbency or trying to |
| | | reach sternal recumbency, responsive |
| | | to sound and painful stimuli. |
| 3 | Moderate sedation | Lateral recumbency, palpebral |
| | | reflexes present, voluntary |
| | | movements (legs, head or ears) |
| | | responsive to sound and painful |
| | | stimuli. |
| 4 | Deep sedation/mild | Lateral recumbency, palpebral |
| | anaesthesia | reflexes present, no voluntary |
| | | movements, unresponsive to sound |
| | | and/or painful stimuli. |
| 5 | Moderate | Lateral recumbency, palpebral |
| | anaesthesia | reflexes present, no voluntary |
| | | movements, unresponsive to sound |
| | | or painful stimuli. |
| 6 | Deep anaesthesia | All reflexes lost, apnoea |

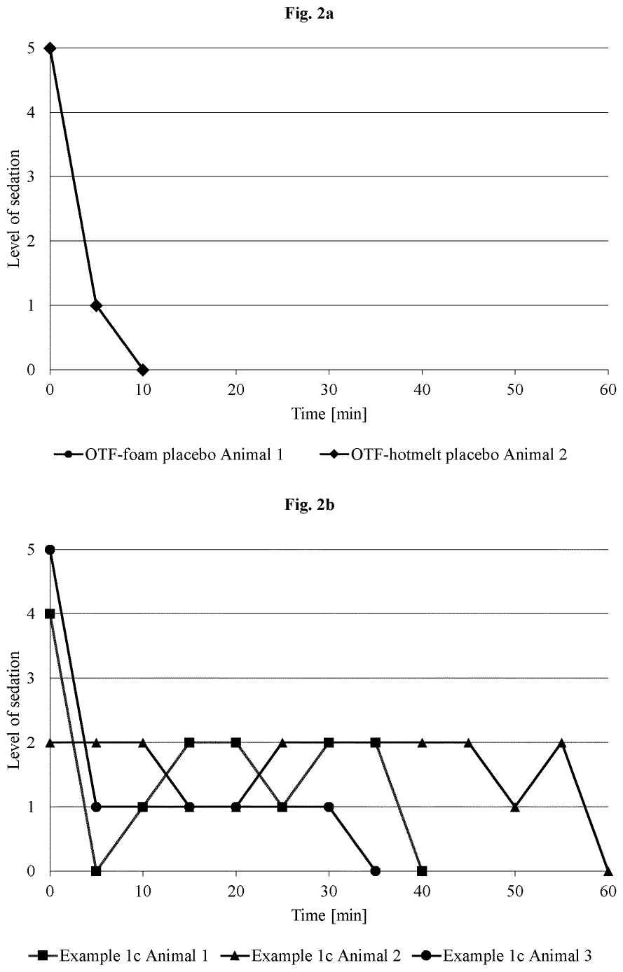

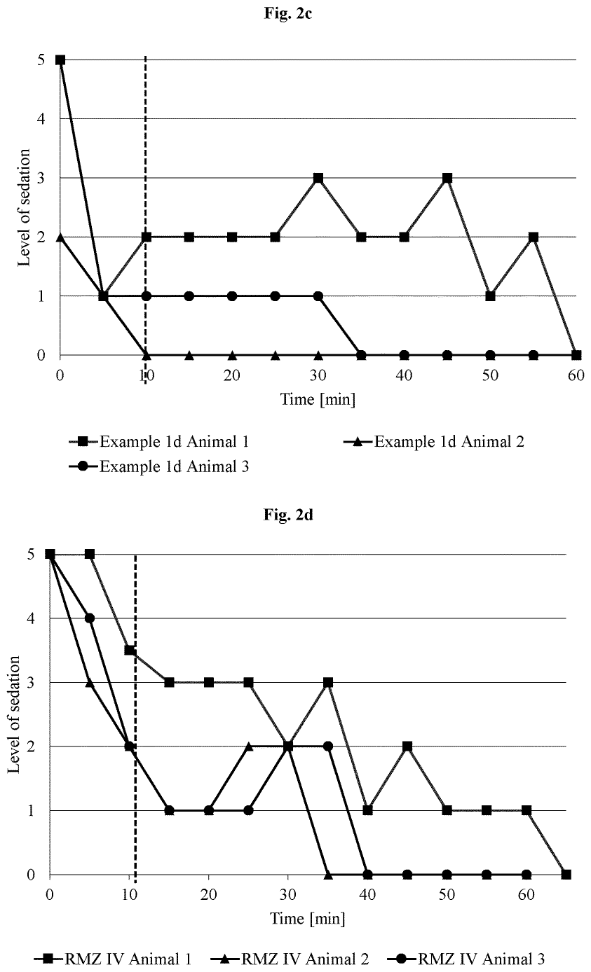

The reflexes and the depth of the anesthesia were monitored every 5 minutes for at least 1 hour and the depth of sedation was characterized as listed in Table 11. The monitoring data is shown in Table 12 for the different Animals and the sedation profiles are illustrated in FIGS. 2a to 2c. The first phase when the animals were dosed with the placebo only shows that the effect of isoflurane wears off within 5-10 minutes. All sedation seen at 10 minutes onwards are therefore considered to be related to the test item.

TABLE 12

| Administration | | | | | | | | | | | | | | |
|---|---|---|---|---|---|---|---|---|---|---|---|---|---|---|
| route, test item | Minutes post dosing | | | | | | | | | | | | | |
| and dose | 0 | 5 | 10 | 15 | 20 | 25 | 30 | 35 | 40 | 45 | 50 | 55 | 60 | 65 |

Animal No 1

| | | | | | | | | | | | | | | |
|---|---|---|---|---|---|---|---|---|---|---|---|---|---|---|
| system-foam, | 5 | 1 | 0 | | | | | | | | | | | |
| Placebo | | | | | | | | | | | | | | |
| IV, RMZ, 2 mL | 5 | 5 | ¾ | 3 | 3 | 3 | 2 | 3 | 1 | 2 | 1 | 1 | 1 | 0 |
| of 5 mg/mL | | | | | | | | | | | | | | |
| system-hotmelt, | 5 | 1 | 2 | 2 | 2 | 2 | 3 | 2 | 2 | 3 | 1 | 2 | 0 | |
| RMZ, 20 mg | | | | | | | | | | | | | | |
| system-foam, | 4 | 0 | 1 | 2 | 2 | 1 | 2 | 2 | 0 | | | | | |
| RMZ, 20 mg | | | | | | | | | | | | | | |

Animal No 2

| | | | | | | | | | | | | | |
|---|---|---|---|---|---|---|---|---|---|---|---|---|---|
| system-hotmelt, | 5 | 1 | 0 | | | | | | | | | | |
| Placebo | | | | | | | | | | | | | |
| IV, RMZ, 2 mL | 5 | 3 | 2 | 1 | 1 | 2 | 2 | 0 | | | | | |
| of 5 mg/mL | | | | | | | | | | | | | |
| system-hotmelt, | 2 | 1 | 0 | | | | | | | | | | |
| RMZ, 20 mg | | | | | | | | | | | | | |
| system-foam, | 2 | 2 | 2 | 1 | 1 | 2 | 2 | 2 | 2 | 2 | 1 | 2 | 0 |
| RMZ, 20 mg | | | | | | | | | | | | | |

Animal No 3

| | | | | | | | | | |
|---|---|---|---|---|---|---|---|---|---|
| ½ system-foam | 5 | 0 | | | | | | | |
| and ½ system- | | | | | | | | | |
| hotmelt, | | | | | | | | | |
| Placebo | | | | | | | | | |
| IV, RMZ, 2 mL | 5 | 4 | 2 | 1 | 1 | 1 | 2 | 2 | 0 |
| of 5 mg/mL | | | | | | | | | |
| system-hotmelt, | 5 | 1 | 1 | 1 | 1 | 1 | 1 | 0 | |
| RMZ, 20 mg | | | | | | | | | |
| system-foam, | 5 | 1 | 1 | 1 | 1 | 1 | 1 | 0 | |
| RMZ, 20 mg | | | | | | | | | |

For the IV administration, the effect of the test item took over directly from the isoflurane, i.e. no fall in sedation followed by an increase was seen. For the hotmelt oromucosal delivery system, a high variability between the animals was observed. An effect was seen in two of three animals as animal No 2 showed no sedation after 10 minutes, i.e. by when the isoflurane effect ended. In the two animals showing a RMZ-related effect, maximal sedation scores of 1 and 3 were observed and full recovery was noted not before 35 and 60 minutes, respectively. For the foam oromucosal delivery system, less variability between animals was observed as compared to the hot melt variant. An effect was seen in all three animals, reaching a maximal score of 2 in two animals and score 1 in one. Full recovery from RMZ-related effects was noted not before 35, 40 and 60 minutes after positioning of the oromucosal delivery system in the three animals.

After each dosing in phase 2-4, blood samples were taken from all animals. Blood samples were collected at 8 time points following application of the oromucosal delivery systems. Samples were collected at the following time points: pre-treatment, and 2, 5, 10, 20, 40, 60 and 90 minutes post-treatment.

RMZ concentrations and CNS7054 concentrations in minipig plasma were determined using a validated liquid-liquid extraction followed by LC-MS/MS. All samples, collected before treatment start, were measured to be below the limit of quantification (0.100 ng/mL).

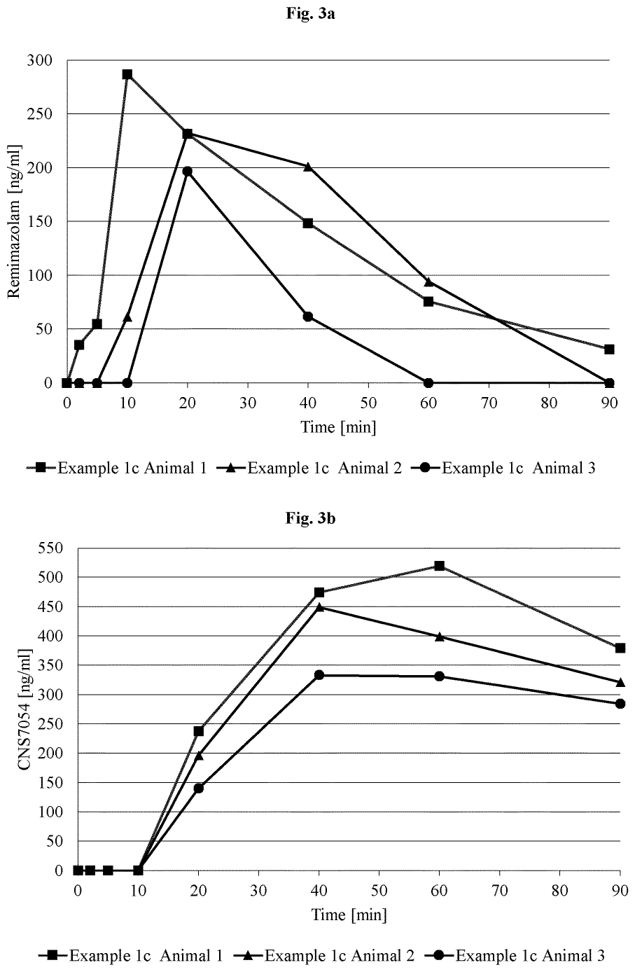

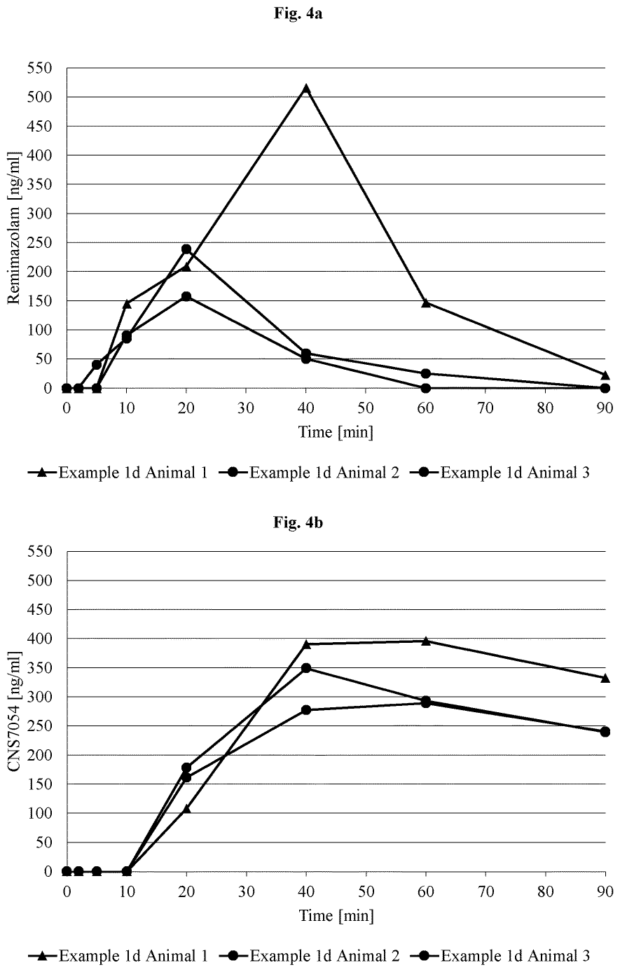

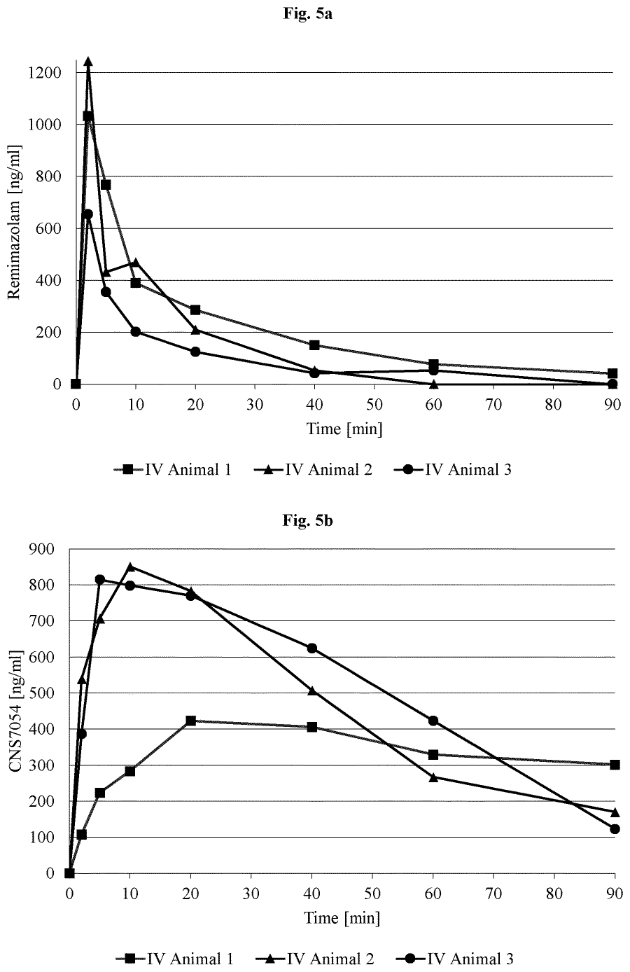

Individual plasma concentrations are listed in Table 13 and are graphically depicted in FIGS. 3a to 5b. Additionally, basic pharmacokinetic parameters such as maximal plasma concentration (cₘₐₓ), time to reaching it (tₘₐₓ) and exposure defined as Area Under the Curve (AUC₀₋ₜ) are summarized in Table 13 for each individual animal for both RMZ and CNS7054 and following IV administration or administration of either of the two oromucosal delivery system formulations.

TABLE 13

| | Remimazolam (ng/ml) | | | CNS7054 (ng/ml) | | |
|---|---|---|---|---|---|---|
| | Animal | Animal | Animal | Animal | Animal | Animal |
| Time (min) | No 1 | No 2 | No 3 | No 1 | No 2 | No 3 |
| | oromucosal delivery system HOT MELT Example 1d (20 mg) | | | | | |
| 0 | NQ | NQ | NQ | NQ | NQ | NQ |
| 1 | NQ | NQ | NQ | NQ | NQ | NQ |
| 5 | NQ | NQ | 40.4 | NQ | NQ | NQ |
| 10 | 145.3 | 90.8 | 85.4 | NQ | NQ | NQ |
| 20 | 209.1 | 157.5 | 238.8 | 108 | 161 | 178 |
| 40 | 516.1 | 50.1 | 59.7 | 390 | 277 | 349 |
| 60 | 146.9 | NQ | 25.1 | 396 | 289 | 293 |
| 90 | 22.5 | NQ | NQ | 332 | 240 | 239 |
| cₘₐₓ (ng/ml) | 516.1 | 157.5 | 238.8 | 396 | 283 | 349 |
| tₘₐₓ (min) | 40 | 20 | 20 | 60 | 60 | 40 |
| AUC₀₋ₜ (min*ng/ml) | 18558.25 | 4045.5 | 6205.6 | 24300 | 18630 | 20560 |
| | oromucosal delivery system FOAM Example 1c (20 mg) | | | | | |
| 0 | NQ | NQ | NQ | NQ | NQ | NQ |
| 1 | 35.3 | NQ | NQ | NQ | NQ | NQ |
| 5 | 54.7 | NQ | NQ | NQ | NQ | NQ |
| 10 | 286.9 | 61.6 | NQ | NQ | NQ | NQ |
| 20 | 231.7 | 232.4 | 196.6 | 237 | 196 | 140 |
| 40 | 148.5 | 201.2 | 61.4 | 474 | 449 | 333 |
| 60 | 75.6 | 94.3 | NQ | 519 | 399 | 331 |
| 90 | 31.3 | NQ | NQ | 379 | 321 | 284 |
| cₘₐₓ (ng/ml) | 286.9 | 232.4 | 196.6 | 519 | 449 | 333 |
| tₘₐₓ (min) | 10 | 20 | 20 | 60 | 40 | 40 |
| AUC₀₋ₜ (min*ng/ml) | 11263.8 | 10329.5 | 4177 | 31695 | 26710 | 21295 |
| | IV (10 mg) | | | | | |
| 0 | NQ | NQ | NQ | NQ | NQ | NQ |
| 1 | 1032 | 1244 | 654 | 108 | 539 | 387 |
| 5 | 767 | 432 | 353.9 | 224 | 707 | 815 |
| 10 | 389.5 | 469 | 201 | 283 | 851 | 798 |
| 20 | 285.5 | 208.8 | 124.3 | 423 | 783 | 770 |
| 40 | 149.3 | 52.6 | 41.2 | 406 | 508 | 625 |
| 60 | 76.1 | NQ | 52.2 | 329 | 267 | 423 |
| 90 | 40.6 | NQ | NQ | 302 | 170 | 123 |
| cₘₐₓ (ng/ml) | 1032 | 1244 | 654 | 423 | 851 | 815 |
| tₘₐₓ (min) | 2 | 2 | 2 | 20 | 10 | 5 |
| AUC₀₋ₜ (min*ng/ml) | 18349.25 | 12539.5 | 8551.6 | 30508.5 | 41688 | 46682.5 |

NQ—not quantifiable (set to 0 for PK analysis);
AUC₀₋ₜ was calculated using the trapezoid method

The two oromucosal delivery system formulations were largely comparable in both, RMZ plasma pharmacokinetics (PK) and cₘₐₓ. The hot-melt formulation of Example 1d shows a higher variability in the PK profiles of RMZ, however, this is to some extent drive by results observed in Animal No 1, where a higher cₘₐₓ is reached at a later time point. Oromucosal delivery system foam formulations are associated with a faster release (tₘₐₓ: 10-20 min for foam vs 20-40 min for hot melt) and a more uniform PK profile compared to the oromucosal delivery system hot melt formulation. However, this conclusion is based on a small animal number only. PK profiles for RMZ's main metabolite, CNS7054, were in line with what was observed for RMZ, reaching cₘₐₓ slightly faster with the oromucosal delivery system foam formulation than with the oromucosal delivery system hot melt formulation. This, together with a slightly higher reached cₘₐₓ, results in a trend to higher exposures to the metabolite when an oromucosal delivery system foam formulation is administered as when an oromucosal delivery system hot melt formulation is administered.

For the evaluation of the bioavailability (BA), dose-normalized cₘₐₓ (cₘₐₓ/D) and dose-normalized AUC (AUC₀₋ₜ/D) were determined for the two oromucosal delivery system formulations for RMZ and CNS7054. The results are summarized in Table 14.

TABLE 14
Remimazolam

| | Example 1d | | Example 1c | | IV | | Example 1d | | Example 1c | |
|---|---|---|---|---|---|---|---|---|---|---|
| | cₘₐₓ/D (ng/ml * mg) | | | | | | cₘₐₓ BA (%) | | | |
| Animal No 1 | 25.81 | | 14.35 | | 103.20 | | 25 | | 14 | |
| Animal No 2 | 7.88 | | 11.62 | | 124.40 | | 6 | | 9 | |
| Animal No 3 | 11.94 | | 9.83 | | 65.40 | | 18 | | 15 | |
| Mean (SD) | 15.21 | (9.4) | 11.93 | (2.27) | 97.67 | (29.89) | 17 | (9) | 13 | (3) |
| | AUC₀₋ₜ/D (min * ng/ml * mg) | | | | | | AUV₀₋ₜ BA (%) | | | |
| Animal No 1 | 927.9 | | 563.2 | | 1834.9 | | 51 | | 31 | |
| Animal No 2 | 202.3 | | 516.5 | | 1254.0 | | 16 | | 41 | |
| Animal No 3 | 310.3 | | 208.9 | | 855.2 | | 36 | | 24 | |
| Mean (SD) | 480.2 | (391.5) | 429.5 | (192.5) | 1314.7 | (492.7) | 34 | (17) | 32 | (8) |

CNS7054

| | | | | | | | cₘₐₓ/D (oromucosal delivery | | | |
|---|---|---|---|---|---|---|---|---|---|---|
| | cₘₐₓ/D (ng/ml * mg) | | | | | | system vs IV) ratio (%) | | | |
| Animal No 1 | 19.8 | | 25.95 | | 42.3 | | 47 | | 61 | |
| Animal No 2 | 14.15 | | 22.45 | | 42.3 | | 17 | | 26 | |
| Animal No 3 | 17.45 | | 16.65 | | 81.5 | | 21 | | 20 | |
| Mean (SD) | 17.13 | (2.84) | 21.68 | (4.7) | 69.63 | (23.74) | 28 | (16) | 36 | (22) |
| | | | | | | | AUC₀₋ₜ/D (oromucosal delivery | | | |
| | AUC₀₋ₜ/D (min * ng/ml * mg) | | | | | | system vs IV) ratio (%) | | | |
| Animal No 1 | 1215 | | 1585 | | 1825 | | 80 | | 104 | |
| Animal No 2 | 932 | | 1336 | | 2084 | | 45 | | 64 | |
| Animal No 3 | 1028 | | 1065 | | 2334 | | 44 | | 46 | |
| Mean (SD) | 1058 | (144) | 1328 | (260) | 1981 | (414) | 56 | (20) | 71 | (30) |

The cₘₐₓ-based BA of RMZ ranged between 6-25% for the hot melt oromucosal delivery system formulation of Example 1d and between 9-25% for the foam oromucosal delivery system formulation of Example 1c. Corresponding results for AUC-based BA were 16-51% and 24-41%. Although the mean exposure parameters were similar for the two RMZ oromucosal delivery system formulations, the higher variability observed following the application of the hot melt oromucosal delivery system formulation of Example 1d may point to its lower adhesion potential to the oral mucosa, that may lead to detachment of the film from the application site and occasional and unpredictable swallowing of the drug. Consequently, reduced BA can be observed due to extensive first pass elimination of the portion of the dose swallowed. Relative exposure to CNS7054 was overall higher following administration of the oromucosal delivery system foam formulation of Example 1c, which is consistent with a faster uptake compared to the oromucosal delivery system hot melt formulation of Example 1d. Taken together, the PK results show a highly promising bioavailability (BA) of remimazolam when administered as an oromucosal delivery system.

### Examples 2A-2B

### Preparation of the Coating Composition (Active Agent-Containing Layer) and Coating of the Coating Composition

The formulations for Examples 2a-2b are summarized in Table 15.

For the example 2a, a beaker was loaded with remimazolam besylate and aqua purificata. The mixture was manually stirred until the mixture was homogeneous. Then Kollicoat IR was added and the mixture was again manually stirred until the mixture was homogenous. PVA 40-88 and aqua purificata were added to the mixture and it was again manually stirred until the mixture was homogenous followed by stirring at 220 rpm for 50 min and foaming of the mixture by stirring at 2000 rpm for 4 min.

The resulting active agent-containing composition was coated on Polyslik 111/80 and was dried at 70° C. for 25 min.

For the example 2b, a beaker was loaded with ascorbic acid, sucralose, saccharin-Na and aqua purificata. The mixture was stirred until all sweeteners were dissolved followed by the addition of remimazolam besylate. The mixture was manually stirred until the mixture was homogenous followed by the addition of Kollicoat IR and manually stirring until the mixture was homogenous. Then, a mixture of PVA 40-88 and aqua purificata was added and the composition was manually stirred until the mixture was homogenous followed by stirring at 220 rpm for 50 min and foaming of the mixture by stirring at 2000 rpm for 4 min.

The resulting active agent-containing composition was coated on Polyslik 111/80 and was dried at 70° C. for 25 min.

TABLE 15

| | Example | | | |
|---|---|---|---|---|
| | 2a | | 2b | |
| | Solids | Amt | Solids | Amt |
| | [%] | [g] | [%] | [g] |
| Ingredients | | | | |
| RMZ besylate | 56.6 | 28.9 | 56.69 | 14.45 |
| PVA 40-88 | 10.8 | 5.53 | 10.08 | 2.57 |
| Kollicoat IR | 32.5 | 16.59 | 30.21 | 7.70 |
| Sucralose | — | — | 2.00 | 0.51 |
| Saccharin-Na | — | — | 0.98 | 0.25 |
| Ascorbic acid | — | — | 0.04 | 0.01 |
| Solvent | | | | |
| Aqua purificata | — | 99.2 | — | 49.5 |
| solids content | 100 | 51.02 | 100 | 25.49 |

film characteristics

| | | |
|---|---|---|
| Area weight [g/m²] | 165 | 122 |
| RMZ-besylate content [mg/system] | 56.1 | 41.5 |
| system size [cm²] | 6 | 6 |

### Preparation of the Oromucosal Delivery System (Concerning Examples 2a & 2b)

Individual oromucosal delivery systems were then punched out from the active agent-containing layer. The oromucosal delivery systems were then sealed into water steam proof Surlyn® pouches without a water absorber patch or together with two water absorber patches (DesiMax).

### Stability Study to Evaluate the Stability of the Remimazolam Besylate in the Different Formulations

This stress stability study for the examples 2a and 2b was performed at 60° C. with measurement timepoints at 2 weeks (2 W), 6 weeks (6 W) and 9 weeks (9 W). The 9 W was set to extrapolate to 2 years real-time under estimation of Arrhenius kinetics.

For each measurement timepoint, a total quantity of 9 samples were provided including 3 samples for determination of water content via Karl-Fischer and 6 samples for analysis of degradation products and assay (n=3 for testing) of which 3 samples are intended as back-up.

The results of the stability study for the examples 2a and 2b are shown in Table 16 and 17.

TABLE 16

| | Example 2a | | | | | | | |
|---|---|---|---|---|---|---|---|---|
| | Condition: | | | | | | | |
| | Packaging in Surlyn ® pouches | | | | | | | |
| | | Water | Impurities (%) | | | | | |
| | Assay | content | | | Unk | Unk | Unk | |
| Storage | (mg/system)/ | (%)/RSD | CNS | CNS | RRT | RRT | RRT | |
| time | RSD (%) | (%) | 7054 | 7084 | 0.34 | 0.36 | 1.21 | Total |
| Initial* | 59.2 | 2.25 | 0.13 | n.d. | n.d. | n.d. | n.d. | 0.26 |
| | (5.0) | (7.4) | | | | | | |
| Sub- | w/o water absorber | | | | | | | |
| Condition: | | | | | | | | |

60° C.

| | | | | | | | | |
|---|---|---|---|---|---|---|---|---|
| 2 weeks | 56.8 | 2.10 | 0.27 | 0.19 | n.d. | n.d. | 0.17 | 0.79 |
| | (2.3) | (3.9) | | | | | | |
| 6 weeks | 57.0 | 1.98 | 0.38 | 0.52 | 0.11 | 0.06 | 0.29 | 1.65 |
| | (3.6) | (3.3) | | | | | | |

| | |
|---|---|
| 9 weeks | n.t.** |

25° C./60% r.h.

| 6 months^{#} | n.t. | n.t. | 0.19 | n.d. | n.d. | n.d. | 0.16 | 0.48 |
|---|---|---|---|---|---|---|---|---|
| Sub- | with water absorber | | | | | | | |
| Condition: | | | | | | | | |

60° C.

| | | | | | | | | |
|---|---|---|---|---|---|---|---|---|
| 2 weeks | 59.1 | 0.79 | 0.11 | 0.06 | n.d. | <0.05 | <0.05 | 0.23 |
| | (4.6) | (48.0) | | | | | | |
| 6 weeks | 57.7 | 0.66 | 0.11 | 0.15 | n.d. | n.d. | n.d. | 0.38 |
| | (2.1) | (26.4) | | | | | | |
| 9 weeks | 56.0 | 0.33 | 0.11 | 0.19 | n.d. | n.d. | n.d. | 0.41 |
| | (4.7) | (7.3) | | | | | | |

25° C./60% r.h.

| | | | | | | | | |
|---|---|---|---|---|---|---|---|---|
| 5 months | n.t. | n.t. | 0.11 | n.d. | n.d. | n.d. | n.d. | 0.24 |

*Initial values were determined without water absorber.
**Only testing of samples with water adsorber was continued.

TABLE 17

| | Example 2b | | | | | | | |
|---|---|---|---|---|---|---|---|---|
| | Condition: | | | | | | | |
| | Packaging in Surlyn ® pouches | | | | | | | |
| | Assay | | | | | | | |
| | (mg/oromucosal | Water | Impurities (%) | | | | | |
| | delivery | content | | | Unk | Unk | Unk | |
| Storage | system)/RSD | (%)/RSD | CNS | CNS | RRT | RRT | RRT | |
| time | (%) | (%) | 7054 | 7084 | 0.34 | 0.36 | 1.21 | Total |
| Initial | 43.7 | 2.50* | 0.14 | n.d. | n.d. | n.d. | n.d. | 0.22 |
| | (1.6) | (9.7) | | | | | | |
| Sub- | w/o water absorber | | | | | | | |
| Condition: | | | | | | | | |

60° C.

| | | | | | | | | |
|---|---|---|---|---|---|---|---|---|
| 2 weeks | 41.5 | 2.28 | 0.44 | 1.31 | 0.14 | 0.10 | 0.18 | 2.26 |
| | (1.2) | (13.0) | | | | | | |

| | |
|---|---|
| 6 weeks | n.t .** |
| 9 weeks | n.t.** |

25° C./60% r.h.

| 5 months | n.t.** |
|---|---|
| Sub- | with water absorber |
| Condition: | |

60° C.

| | | | | | | | | |
|---|---|---|---|---|---|---|---|---|
| 2 weeks | 44.0 | 0.74 | 0.10 | 1.21 | n.d. | n.d. | n.d. | 1.37 |
| | (5.3) | (23.7) | | | | | | |

| | |
|---|---|
| 6 weeks | n.t.** |
| 9 weeks | n.t.** |

25° C./60% r.h.

| | | | | | | | | |
|---|---|---|---|---|---|---|---|---|
| 5 months | n.t. | n.t. | 0.11 | 0.40 | n.d. | n.d. | n.d. | 0.65 |

*Initial values were determined without water absorber.
**Discontinued due to high degradation.
n.t. = not tested

The stress stability study showed that the Example 2a (basic formulation without sweetener and antioxidant) produced via a water-based production process is only stable in terms of degradation when pouched together with water absorbers. Arrhenius kinetics assumed; all samples of basis formulation pouched together with water absorbers could potentially be stable for at least 2 years at 25° C.

This assumption can be supported by the results obtained for the samples stored at 25° C. over 5 months and 6 months since the level of known degradation products CNS 7054 (in samples without water adsorber) and CNS 7084 was lower than the level obtained for the equivalent samples (following Arrhenius extrapolation) stored at 60° C. for 2 weeks. In this regard, the degradation profile of samples stored with water absorber at 25° C. for 2 years is expected to show less degradation products than implied by the 9 weeks/60° C. data of this stress stability study. However, an official shelf-life indicating stability study (e.g., storage temperatures 25° C. and 40° C. following ICH) is needed to determine a concrete shelf-life for each formulation.

At relevant concentrations, for the different unknown impurities which are known to be formed partly by oxidation reaction and are therefore temperature induced, lower degradation profiles are expected at long term conditions compared to the equivalent sampling timepoints at 60° C.

Example 2b with sucralose, saccharine-Na and ascorbic acid as sweeteners and antioxidant respectively, showed high degradation for both conditions (with and without water absorber) already after 2 weeks at 60° C. The high degradation was accompanied by very significant and intense brownish to dark beige discolorations. Although CNS 7054 was very low and the formation of the unknown impurities was completely suppressed in samples with water absorber after 2 weeks, the concentration of CNS 7084 was remarkably high, which could be related to incompatibilities with the contained sweeteners and/or antioxidant. The effect of the antioxidant ascorbic acid could not be demonstrated with Example 2b.

### Mucosa Permeation Study

For the investigation of the potential impact of pH on the permeation, a permeation study was performed to assess the permeation of remimazolam besylate (RMZ besylate) through the mucosa in dependence of pH value. In a second run, also the performance of remimazolam lyophilizate product (RMZ besylate) vs. oromucosal delivery system was assessed.

The permeation settings are summarized in Table 18. The samples used for permeation run 1 are defined in Table 19.

TABLE 18

| Mucosa | Temperature | Acceptor | Sampling Times (min) |
|---|---|---|---|
| Pig Esophagus, | 37° C. | Phosphate | 5-10-15-20- |
| 400 μm | | Buffer pH 7.4 | 25-30-60 |

TABLE 19

| | | Resulting | Set | Appearance of |
|---|---|---|---|---|
| Sample | Medium | pH | pH | Donor Solution |
| Example 2a | Artificial Saliva | 3.65 | — | Clear solution |
| Example 2a | 0.9% NaCl | 3.54 | — | Clear solution |
| RMZ | Artificial Saliva | — | 2 | Clear solution |
| RMZ | Artificial Saliva | — | 3 | Little RMZ solid |
| | | | | residuals |
| RMZ | Artificial Saliva | — | 4.5 | Much RMZ solid |
| | | | | residuals |
| RMZ | Artificial Saliva | — | 6 | Very much RMZ solid |
| | | | | residuals |

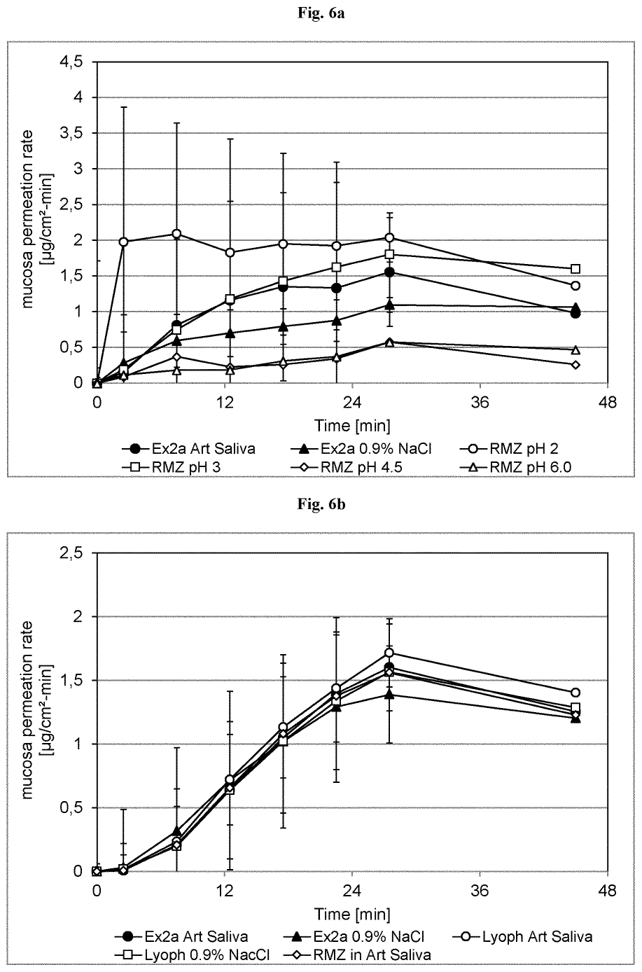

The permeated amount and the corresponding mucosa permeation rates of the oromucosal delivery system prepared according to Examples 2a were determined by in vitro experiments in accordance with the OECD Guideline (adopted Apr. 13, 2004) using pig mucosa with a thickness of 400 μm (mucosa oesophagus). The donor solutions were prepared by dissolving a sample film of Example 2a with a size of 5.75 cm² either in 5 ml artificial saliva or in 5 ml 0.9% NaCl. The remimazolam besylate was dissolved in artificial saliva so that the applied dose of RMZ was equivalent to an oromucosal delivery system containing 50 mg remimazolam in 500 μL medium, and the pH of the mixture was adjusted as indicated in Table 19. The pH was measured using a pH electrode. The permeation donor solutions were directly applied to the mucosa (diffusion area of 4.524 cm²). The remimazolam permeated amount in the receptor medium (phosphate buffer solution pH 7.4) at a temperature of 37±1° C. was measured and the corresponding mucosa permeation rate calculated. The results are shown in Table 20 and FIG. 6a.

TABLE 20

| | mucosa permeation rate with SD [μg/cm² min] (for n =3 ) | | | | | | |
|---|---|---|---|---|---|---|---|
| Sample | 2.5 min | 7.5 min | 12.5 min | 17.5 min | 22.5 min | 27.5 min | 45 min |
| 2a in | 0.15 | 0.81 | 1.16 | 1.35 | 1.33 | 1.56 | 0.98 |
| artificial | (±0.0784) | (±0.8060) | (±1.2083) | (±1.3859) | (±1.3173) | (±1.4824) | (±0.7619) |
| saliva | | | | | | | |
| 2a in | 0.28 | 0.59 | 0.70 | 0.79 | 0.88 | 1.10 | 1.06 |
| 09% | (±0.2154) | (±0.4373) | (±0.3678) | (±0.3274) | (±0.2486) | (±0.2911) | (±0.1012) |
| NaCl | | | | | | | |
| RMZ in | 1.98 | 2.09 | 1.83 | 1.95 | 1.92 | 2.04 | 1.36 |
| artificial | (±1.7137) | (±1.8866) | (±1.5489) | (±1.5914) | (±1.2725) | (±1.1734) | (±0.3431) |
| saliva | | | | | | | |
| (pH 2) | | | | | | | |
| RMZ in | 0.19 | 0.75 | 1.18 | 1.43 | 1.62 | 1.80 | 1.60 |
| artificial | (±0.1488) | (±0.2276) | (±0.2523) | (±0.3930) | (±0.4128) | (±0.5894) | (±0.5171) |
| saliva | | | | | | | |
| (pH 3) | | | | | | | |
| RMZ in | 0.09 | 0.37 | 0.22 | 0.26 | 0.34 | 0.58 | 0.26 |
| artificial | (±0.1459) | (±0.6125) | (±0.2881) | (±0.2611) | (±0.2740) | (±0.2913) | (±0.0313) |
| saliva | | | | | | | |
| (pH 4.5) | | | | | | | |
| RMZ in | 0.11 | 0.18 | 0.18 | 0.31 | 0.37 | 0.58 | 0.47 |
| artificial | (±0.1706) | (±0.1640) | (±0.1118) | (±0.1805) | (±0.1890) | (±0.1780) | (±0.3066) |
| saliva | | | | | | | |
| (pH 6) | | | | | | | |

In permeation experiment with RMZ dissolved in artificial saliva at different pH values (pH 2, 3, 4.5, 6), a distinct dependency between solubility and pH value was confirmed, which showed, that a decrease of pH results in an increase in permeability. At pH 4.5 and pH 6, the permeation rate was poor due to low solubility of RMZ.

At pH 2, RMZ was dissolved completely. In this run, only 2 out of 3 wells could be analyzed since one well barrier function was compromised due to contracted mucosa. The permeation values of the remaining samples diverged very significantly from each other, so that it can be assumed that the barrier function of the highly permeable mucosa was also compromised. From the results it is currently not possible to determine whether the strong reduction of the pH leads to an improvement in permeation. Nevertheless, due to the harsh acidic pH 2, a pH adjustment to that low pH in oromucosal delivery system samples is not favorable in the in-vivo situation.

Due to the high variability in permeation run 1 (n=3), a second permeation experiment was performed with more replicates (n=6) to enlarge the data basis. The permeation settings are summarized in Table 21. The samples used are defined in table 15. Example 2a and Lyophilizate were dissolved in artificial saliva and 0.9% NaCl, respectively. Due to a pH adjustment in the lyophilizate/drug product, the resulting pH was slightly lower (pH 3.14 and pH 3.09) than with the Example 2a without pH adjustment (pH 3.67 and pH 3.50). A RMZ sample set to pH 3 served as a control.

TABLE 21

| | | Resulting | Set | Appearance of |
|---|---|---|---|---|
| Sample | Medium | pH | pH | Donor Solution |
| Example 2a | Artificial | 3.67 | — | Clear Solution |
| | Saliva | | | |
| Example 2a | 0.9% NaCl | 3.50 | — | Clear Solution |
| Lyophilizate/ | Artificial | 3.14 | — | Clear Solution |
| drug product | Saliva | | | |
| Lyophilizate/ | 0.9% NaCl | 3.09 | — | Clear Solution |
| drug product | | | | |
| RMZ | Artificial | — | 3 | Little RMZ solid |
| | Saliva | | | residuals |

The permeated amount and the corresponding mucosa permeation rates of the oromucosal delivery system prepared according to Examples 2a were determined as outlined above. The remimazolam permeated amount in the receptor medium (phosphate buffer solution pH 7.4) at a temperature of 37±1° C. was measured and the corresponding mucosa permeation rate calculated. The results are shown in Table 22 and FIG. 6b.

TABLE 22

| | mucosa permeation rate with SD [μg/cm² min] (for n = 6) | | | | | | |
|---|---|---|---|---|---|---|---|
| Sample | 2.5 min | 7.5 min | 12.5 min | 17.5 min | 22.5 min | 27.5 min | 45 min |
| 2a in | 0.01 | 0.21 | 0.64 | 1.05 | 1.40 | 1.60 | 1.25 |
| artificial | (±0.0164) | (±0.2118) | (±0.4409) | (±0.5392) | (±0.5883) | (±0.5955) | (±0.3403) |
| saliva | | | | | | | |
| 2a in | 0.03 | 0.32 | 0.72 | 1.02 | 1.29 | 1.39 | 1.20 |
| 09% | (±0.0612) | (±0.4593) | (±0.6554) | (±0.7006) | (±0.6794) | (±0.5903) | (±0.3819) |
| NaCl | | | | | | | |
| Lyoph. in | 0.02 | 0.23 | 0.72 | 1.13 | 1.44 | 1.72 | 1.40 |
| artificial | (±0.0096) | (±0.1154) | (±0.2790) | (±0.3555) | (±0.3974) | (±0.4200) | (±0.2687) |
| saliva | | | | | | | |
| Lyoph. in | 0.02 | 0.20 | 0.64 | 1.02 | 1.34 | 1.57 | 1.29 |
| 0.9% | (±0.0139) | (±0.0944) | (±0.2542) | (±0.3405) | (±0.4010) | (±0.4069) | (±0.2546) |
| NaCl | | | | | | | |
| RMZ in | 0.01 | 0.21 | 0.65 | 1.08 | 1.38 | 1.56 | 1.23 |
| artificial | (±0.0073) | (±0.1311) | (±0.3286) | (±0.4969) | (±0.5750) | (±0.6156) | (±0.4345) |
| saliva | | | | | | | |
| (pH 3) | | | | | | | |

The mucosa permeation data (see FIG. 6b) show no significant difference between Example 2a and the lyophilizate drug product samples (both 0.900 NaCl and artificial saliva). RMZ samples at pH 3 in artificial saliva show no significant difference to Example 2a and lyophilizate drug product samples (both 0.9% NaCl and artificial saliva).

### Examples 2C-2K

### Preparation of the Coating Composition (Active Agent-Containing Layer) and Coating of the Coating Composition

The formulations for the Examples 2c-2j (foam formulations) are summarized in the Table 24.

For the examples 2c, 2f, a beaker was loaded with the corresponding sweetener and aqua purificata. The mixture was manually stirred until the sweetener was dissolved. Remimazolam besylate was added and the mixture was again manually stirred until the mixture was homogenous. For the example 2 f, the mixture was treated in an ultrasonic bath for 30 s for two times after manually stirring as the mixture was not homogenous after manually stirring. Then Kollicoat JR was added followed by manually stirring until the mixture was homogenous. Then PVA 40-88 mixed with aqua purificata was added and the newly formed mixture was stirred until the mixture was homogenous. The mixture was stirred at 200 rpm for 15 min followed by forming the foam by stirring at 2000 rpm for 5 min.

The active agent-containing composition was coated on Polyslik 111/80 (one side siliconized) and dried for 25 min at 70° C.

For the example 2d, a beaker was loaded with the corresponding sweetener and aqua purificata. The mixture was stirred at 230 rpm for 10 min. Remimazolam besylate was added and the mixture at 230 rpm for 5 min. Then Kollicoat IR was added followed by stirring at 230 rpm for 5 min. Then PVA 40-88 mixed with aqua purificata was added and the newly formed mixture was stirred manually until the mixture was homogenous. The mixture was stirred at 200 rpm for 15 min followed by forming the foam by stirring at 2000 rpm for 4.5 min.

The active agent-containing composition was coated on Polyslik 111/80 (one side siliconized) and dried for 25 min at 70° C.

For the example 2e, a beaker was loaded with the corresponding sweetener and aqua purificata. The mixture was treated in an ultrasonic bath for 10 min until the sweetener was dissolved. Remimazolam besylate was added and the mixture was manually stirred followed by treating the mixture in an ultrasonic bath for 30 s for two times. Then Kollicoat IR was added followed by manually stirring until the mixture was homogenous. PVA 40-88 mixed with aqua purificata was added and the newly formed mixture was stirred manually until the mixture was homogenous. The mixture was stirred at 200 rpm for 15 min followed by forming the foam by stirring at 2000 rpm for 4.5 min.

The active agent-containing composition was coated on Polyslik 111/80 (one side siliconized) and dried for 25 min at 70° C.

For the examples 2g-2i, a beaker was loaded with the corresponding sweetener and aqua purificata. The mixture was manually stirred followed by treating in an ultrasonic bath for 10 min in the case of the examples 2g, for 30 s for two times with manually stirring between the ultrasonic treatment in the case of example 2 h and for 20 s in the case of example 2i. Remimazolam besylate was added and the mixture was manually stirred followed by treating the mixture in an ultrasonic bath for 30 s for two times with manually stirring between the ultrasonic treatment. Then Kollicoat IR was added followed by manually stirring until the mixture was homogenous. PVA 40-88 mixed with aqua purificata was added and the newly formed mixture was stirred manually until the mixture was homogenous. The mixture was stirred at 200 rpm for 15 min followed by forming the foam by stirring at 2000 rpm for 4.5 min (2g-2h) and 3.75 min (2i).

The active agent-containing composition was coated on Polyslik 111/80 (one side siliconized) and dried for 25 min at 70° C.

For the example 2j a beaker was loaded with the corresponding sweetener and aqua purificata. Remimazolam besylate was added and the mixture was manually stirred followed by treating the mixture in an ultrasonic bath for 30 s for two times. Then Kollicoat IR was added followed by manually stirring until the mixture was homogenous. PVA 40-88 mixed with aqua purificata was added and the newly formed mixture was stirred manually until the mixture was homogenous. The mixture was stirred at 200 rpm for 15 min followed by forming the foam by stirring at 2000 rpm for 5 min.

The active agent-containing composition was coated on Polyslik 111/80 (one side siliconized) and dried for 25 min at 70° C.

The formulation for the example 2k (hot melt formulation) is summarized in Table 23.

For the example 2k, a mortar was loaded with Polyox N10, RMZ besylate, Neotam and Tutti Frutti and the solids were pestled to obtain a homogenous powder. The obtained powder was weighed (171.85 mg per system) and transferred to the vacuum compression molding (VCM) tool. The VCM tool was heated until a homogeneous hotmelt oromucosal delivery system is obtained.

### Preparation of the Oromucosal Delivery System (Concerning all Examples)

The Individual oromucosal delivery systems were then sealed into water steam proof Surlyn® pouches together with two water absorber patches (DesiMax).

TABLE 23

| | |
|---|---|
| Solids [%] | Amt [mg] |

Ingredient (Trade Name)

| | | |
|---|---|---|
| RMZ besylate | 32.38 | 55.65 |
| Polyox N10 | 66.7 | 115.25 |
| Neotam | 0.052 | 0.09 |
| Tutti Frutti | 0.5 | 0.86 |

Film characteristics

| | |
|---|---|
| Area weight [g/m²] | 350 |
| RMZ-besylate content [mg/system] | 55.53 |
| System size [cm²] | 4.9 |

### Stability Study to Evaluate the Stability of the Remimazolam Besylate in the Examples 2C-2K

Due to the incompatibility of RMZ with one or both sweeteners of the formulation of Example 2b (sucralose and saccharin-Na), a second short-term stress stability study was conducted to verify compatible sweeteners for the RMZ-oromucosal delivery system formulation.

The stress stability study for the examples 2C-2K was performed at 60° C. with measurement timepoints at 2 weeks (2 W) and 6 weeks (6 W) for the examples 2c-2j and at 2 weeks (2 W), 6 weeks (6 W) and 9 weeks (9 W) for example 2k.

The results of the stress stability study for the examples 2c-2j are summarized in Table 25.

The results of the stress stability study for the example 2k are summarized in Table 26.

TABLE 24

| | 2c | | 2d | | 2e | | 2f | | 2g | | 2h | | 2i | | 2j | |
|---|---|---|---|---|---|---|---|---|---|---|---|---|---|---|---|---|
| Ingredient | Foam | | Foam | | Foam | | Foam | | Foam | | Foam | | Foam | | Foam | |
| (Trade | Solids | Amt | Solids | Amt | Solids | Amt | Solids | Amt | Solids | Amt | Solids | Amt | Solids | Amt | Solids | Amt |
| Name) | [%] | [g] | [%] | [g] | [%] | [g] | [%] | [g] | [%] | [g] | [%] | [g] | [%] | [g] | [%] | [g] |
| RMZ | 56.62 | 14.45 | 56.74 | 14.46 | 56.57 | 14.43 | 56.63 | 14.45 | 56.62 | 14.46 | 56.65 | 14.44 | 56.64 | 14.46 | 56.71 | 56.67 |
| besylate | | | | | | | | | | | | | | | | |
| PVA 40-88 | 10.65 | 2.72 | 10.26 | 2.61 | 10.55 | 2.69 | 10.87 | 2.77 | 10.76 | 2.75 | 10.79 | 2.75 | 10.68 | 2.73 | 10.51 | 10.53 |
| Kollicoat IR | 31.74 | 8.1 | 31.00 | 7.9 | 31.91 | 8.14 | 32.45 | 8.28 | 32.58 | 8.32 | 32.46 | 8.29 | 32.16 | 8.21 | 31.56 | 31.60 |

Sweeteners

| | | | | | | | | | | | | | | | | |
|---|---|---|---|---|---|---|---|---|---|---|---|---|---|---|---|---|
| Sucralose | 0.98 | 0.25 | — | — | — | — | — | — | — | — | — | — | — | — | — | — |
| Acesulfam- | — | — | 2.00 | 0.51 | — | — | — | — | — | — | — | — | — | — | — | — |
| K | | | | | | | | | | | | | | | | |
| Aspartam | — | — | — | — | 0.98 | 0.25 | — | — | — | — | — | — | — | — | — | — |
| Neotame | — | — | — | — | — | — | 0.05 | 0.01 | — | — | — | — | — | — | — | — |
| Advantame | — | — | — | — | — | — | — | — | 0.04 | 0.01 | — | — | — | — | — | — |
| Thaumatine | — | — | — | — | — | — | — | — | — | — | 0.04 | 0.01 | — | — | — | — |
| Tutti Frutti | — | — | — | — | — | — | — | — | — | — | — | — | 0.52 | 0.13 | — | — |
| Orange | — | — | — | — | — | — | — | — | — | — | — | — | — | — | 1.22 | 0.31 |

Solvent

| | | | | | | | | | | | | | | | | |
|---|---|---|---|---|---|---|---|---|---|---|---|---|---|---|---|---|
| Aqua | n.a. | 50.16 | n.a. | 49.44 | n.a. | 49.49 | n.a. | 49.57 | n.a. | 49.24 | n.a. | 49.43 | | 49.43 | | 49.44 |
| purificata | | | | | | | | | | | | | | | | |
| solid | 100 | 25.52 | 100 | 25.48 | 100 | 25.51 | 100 | 25.52 | 100 | 25.54 | 100 | 25.49 | 100 | 25.53 | 100 | 25.54 |
| content | | | | | | | | | | | | | | | | |

Film characteristics

| Area weight | 117 | 111 | 133 | 143 | 162 | 143 | 129 | 131 |
|---|---|---|---|---|---|---|---|---|
| [g/m²] | | | | | | | | |
| RMZ- | 39.78 | 37.74 | 45.22 | 48.62 | 55.01 | 48.62 | 43.86 | 44.5 |
| besylate | | | | | | | | |
| content | | | | | | | | |
| [mg/system] | | | | | | | | |
| system size | 6 | 6 | 6 | 6 | 6 | 6 | 6 | 6 |
| [cm²] | | | | | | | | |

TABLE 25

| | | | | | Unk | Unk | Unk | Unk | Unk | |
|---|---|---|---|---|---|---|---|---|---|---|
| | | Storage | CNS | CNS | RRT | RRT | RRT | RRT | RRT | |
| | | Time | 7054* | 7084* | 0.34 | 0.36 | 0.61 | 0.72 | 1.21 | Total |
| Ex. | Description | (weeks) | (%) | (%) | (%) | (%) | (%) | (%) | (%) | ** |
| 2a | w/o | Initial | 0.19 | n.d. | n.d. | n.d. | n.d | n.d | n.d. | 0.32 |
| | sweetener | 2 | 0.14 | n.d. | n.d. | n.d. | n.d. | n.d. | n.d | 0.22 |
| | reference | 6 | 0.14 | 0.16 | n.d. | n.d. | <0.05 | n.d. | n.d. | 0.42 |
| 2c | with | Initial | 0.18 | n.d | n.d | n.d. | n.d | n.d | n.d. | 0.33 |
| | sweetener | 2 | 0.14 | n.d. | n.d. | n.d. | n.d. | n.d. | n.d. | 0.21 |
| | Sucralose | 6 | 0.13 | 0.22 | n.d. | n.d. | n.d. | n.d. | n.d. | 0.47 |
| 2d | with | Initial | 0.20 | n.d. | n.d. | n.d. | n.d. | n.d. | n.d. | 0.33 |
| | sweetener | 2 | 0.14 | 1.07 | n.d | n.d. | n.d. | n.d. | n.d | 1.33 |
| | Acesulfame- | 6 | | | | n.t. | | | | |
| | K | | | | | | | | | |
| 2e | with | Initial | 0.19 | n.d. | n.d. | n.d. | n.d. | n.d. | n.d. | 0.33 |
| | sweetener | 2 | 0.14 | 0.59 | n.d. | n.d. | n.d. | n.d. | n.d. | 0.79 |
| | Aspartame | 6 | | | | n.t. | | | | |
| 2f | with | Initial | 0.19 | n.d. | n.d. | n.d. | n.d. | n.d | n.d. | 0.32 |
| | sweetener | 2 | 0.14 | n.d. | n.d | n.d. | n.d. | n.d. | n.d. | 0.21 |
| | Neotame | 6 | 0.14 | 0.15 | n.d. | n.d. | <0.05 | n.d. | n.d. | 0.41 |
| 2g | with | Initial | 0.19 | n.d. | n.d. | n.d. | n.d. | n.d | n.d. | 0.33 |
| | sweetener | 2 | 0.14 | n.d. | n.d. | n.d. | n.d. | n.d | n.d. | 0.21 |
| | Advantame | 6 | 0.14 | 0.16 | n.d. | n.d. | n.d. | n.d. | n.d. | 0.43 |
| 2h | with | Initial | 0.19 | n.d. | n.d. | n.d. | n.d. | n.d. | n.d. | 0.33 |
| | sweetener | 2 | 0.14 | n.d. | n.d. | n.d. | n.d. | n.d | n.d. | 0.26 |
| | Thaumatine | 6 | 0.14 | 0.14 | n.d. | n.d. | <0.05 | 0.06 | n.d. | 0.53 |
| 2i | with flavor | Initial | 0.18 | n.d. | n.d. | n.d. | n.d. | n.d. | n.d. | 0.32 |
| | Tutti Frutti | 2 | 0.15 | n.d. | n.d. | n.d. | n.d | n.d. | n.d | 0.21 |
| | | 6 | 0.14 | 0.14 | n.d. | n.d. | n.d | n.d | n.d. | 0.40 |
| 2j | with flavor | Initial | 0.18 | n.d. | n.d. | n.d. | n.d | n.d | n.d. | 0.33 |
| | Orange | 2 | 0.15 | n.d. | n.d. | n.d. | n.d. | n.d | n.d. | 0.22 |
| | | 6 | 0.15 | 0.16 | n.d. | n.d. | n.d. | n.d. | n.d. | 0.44 |

n.t. = not tested
*known degradation products
** including process/process related impurities and isolated intermediates

The sweeteners Neotame and Advantame as well as the flavors Orange and Tutti Frutti represent good options for further formulation development of foam oromucosal delivery system. The degradation profiles of the respective foam formulations (2f, 2e, 2i and 2j, respectively) did not significantly diverge from the reference formulation of example 2a.

TABLE 26

| | | | | | Unk | Unk | Unk | Unk | Unk | Unk | |
|---|---|---|---|---|---|---|---|---|---|---|---|
| | | Storage | CNS | CNS | RRT | RRT | RRT | RRT | RRT | RRT | |
| | | Time | 7054 * | 7084 * | 0.34 | 0.36 | 0.61 | 0.72 | 0.79 | 1.21 | Total |
| Ex. | Description | (weeks) | (%) | (%) | (%) | (%) | (%) | (%) | (%) | (%) | ** |
| 2k | with | Initial | 0.19 | 0.06 | n.d. | n.d. | n.d. | n.d. | n.d. | n.d. | 0.37 |
| | sweetener | 2 | 0.15 | 0.09 | n.d. | n.d. | n.d. | n.d. | n.d. | n.d. | 0.30 |
| | Neotame | 6 | 0.13 | 0.22 | n.d. | n.d. | <0.05 | 0.07 | 0.05 | n.d. | 0.62 |
| | and flavor | 9 | 0.13 | 0.27 | n.d. | n.d. | <0.05 | 0.09 | 0.06 | <0.05 | 0.72 |
| | Tutti Frutti | | | | | | | | | | |

* known degradation products
** including process/process related impurities and isolated intermediates

The hot-melt formulation on Polyox N80 basis was less stable. Example 2k showed a constant increase of CNS 7084 over time and the formation of several unknown degradation products (still at low levels after 6 weeks).

### Examples 3A-3B

### Preparation of the Coating Composition (Active Agent-Containing Layer) and Coating of the Coating Composition

The formulations for the Examples 3a and 3b are summarized in the Table 27.

TABLE 27

| | Example | | | |
|---|---|---|---|---|
| | 3a | | 3b | |
| | Solids | Amt | Solids | Amt |
| | [%] | [g] | [%] | [g] |
| Ingredients | | | | |
| RMZ besylate | 56.67 | 192.67 | 56.67 | 289.01 |
| PVA 40-88 | 16.91 | 57.49 | 10.70 | 54.58 |
| Kollicoat IR | 25.37 | 86.25 | 32.08 | 163.59 |

Excipients

| | | | | |
|---|---|---|---|---|
| Tutti frutti | 1.00 | 3.41 | 0.50 | 2.55 |
| Neotam | 0.05 | 0.17 | 0.05 | 0.25 |
| Solvent | | | | |
| Aqua purificata | n.a. | 702.83 | n.a. | 849.94 |
| solids content | 100 | 340.0 | 100 | 510.0 |

film characteristics

| | | |
|---|---|---|
| Area weight [g/m²] | 213 | 207 |
| RMZ-besylate content [mg/system] | 120.7 | 117.31 |
| system size [cm²] | 10 | 10 |

For Examples 3a and 3b, a beaker was loaded with Neotam and Tutti Frutti and the excipients were mixed with aqua purificata using an Ultra Turrax IKA® T25 digital on low speed until the excipients were completely dissolved. Remimazolam besylate was added followed by the addition of aqua purificata. After manually stirring, the mixture was homogenized using an Ultra Turrax KA® T25 digital starting at low speed and gradual increased up to 20000 rpm for 60 sec under N₂ atmosphere. Kollicoat IR was added and the mixture was manually stirred with a 4-blade stirrer followed by stirring at low speed for at least 15 in until homogenous under N₂ atmosphere. Then PVA 40-88 pre-solved in aqua purificata was added and the mixture was manually stirred with a 4-blade stirrer at low to medium speed for 30 min under N₂ atmosphere until the mixture was homogenous. The mixture was then stirred at 160 rpm for 25 min followed by by stirring at 100 min for 30 min under N₂ atmosphere.

For the foam formation a foam device was used, which had the following settings for Example 3a:
Feeding speed (hose pump): 150 mL/min (hose type: SPT 3350, inner diameter 6.4 mm, wall thickness 2.4 mm; calibration performed with water)
N₂-flow: 40 min/min
Foaming head rotor speed: 7600 rpm

For the foam formation a foam device was used, which had the following settings for Example 3b:
Feeding speed (hose pump): 110 mL/min (hose type: SPT 3350, inner diameter 6.4 mm, wall thickness 2.4 mm; calibration performed with water)
N₂-flow: 55 min/min
Foaming head rotor speed: 8500 rpm

The first foam flow of every foaming step was discarded. 4 foaming sequences were performed for Example 3a and 3b. In between the foaming sequences, the foam coating composition was stirred at low speed and the preparation vessel was covered.

The active agent-containing foam coating composition was coated on Polyslik 111/80 (one side siliconized) and dried for 25 min at 70° C.

### Preparation of the Oromucosal Delivery System

Individual oromucosal delivery systems were then punched out from the active agent-containing layer in the sizes of 1.5 cm², 3 cm² and 6 cm². The oromucosal delivery systems were then sealed into water steam proof Surlyn® pouches as follows:
I. systems are placed inside the folded liner
   1.5 cm² system: 1 desiccant label (DesiMax®) on the outside of the folded liner
   3 cm² system: 1 desiccant label (DesiMax®) on the outside of the folded liner
   6 cm² system: 2 desiccant labels (DesiMax®) on the outside of the folded liner
II. Folded liner with an oromucosal delivery system and desiccant label(s) is inserted into the pouch with the opening facing down.

The invention relates in particular to the following further embodiments:
1. Oromucosal delivery system for the transmucosal delivery of an active agent, comprising an active agent-containing layer comprising:
   i) remimazolam, a pharmaceutically acceptable salt, or any other form thereof, as active agent and
   ii) a film-forming agent.
2. Oromucosal delivery system according to embodiment 1,
wherein the active agent-containing layer comprises
   at least 20 wt-%, at least 25 wt-% or at least 30 wt-% of the active agent, and/or
   less than or equal to 60 wt-%, less than or equal to 55 wt-%, or less than or equal to 50 wt-% of the active agent, and/or
   from 20 to 60 wt-%, from 25 to 55 wt-% or from 30 to 50 wt-% of the active agent.
3. Oromucosal delivery system according to embodiment 1 or 2,
wherein the active agent-containing layer comprises
   at least 4 mg/cm², at least 6 mg/cm² or at least 8 mg/cm² of the active agent, and/or
   less than or equal to 15 mg/cm², less than or equal to 13 mg/cm², or less than or equal to 11 mg/cm² of the active agent, and/or
   from 4 to 15 mg/cm², from 6 to 13 mg/cm² or from 8 to 11 mg/cm² of the active agent.
4. Oromucosal delivery system according to any one of embodiments 1 to 3,
wherein the oromucosal delivery system comprises
   at least 5 mg, at least 10 mg or at least 15 mg of the active agent, and/or
   less than or equal to 80 mg, less than or equal to 70 mg, or less than or equal to 60 mg of the active agent, and/or
   from 5 to 80 mg, from 10 to 70 mg or from 15 to 60 mg of the active agent.
5. Oromucosal delivery system according to any one of embodiments 1 to 4,
wherein the active agent is remimazolam, a pharmaceutically acceptable salt of remimazolam, or any mixture thereof.
6. Oromucosal delivery system according to embodiment 5,
wherein the active agent is remimazolam besylate or remimazolam tosylate.
7. Oromucosal delivery system according to any one of embodiments 1 to 6,
wherein the active agent in the active agent-containing layer is dissolved or dispersed, or is in the form of non-micronized particles.
8. Oromucosal delivery system according to any one of embodiments 1 to 7 comprising an active agent-containing layer comprising:
   i) remimazolam, a pharmaceutically acceptable salt, or any other form thereof, as active agent,
   ii) a film-forming agent, and
   iii) a plasticizer.
9. Oromucosal delivery system according to embodiment 8,
wherein the plasticizer is selected from the group consisting of linear or branched, saturated or unsaturated alcohols having 6 to 20 carbon atoms, triglycerides, polyethylene glycol or polyvinyl alcohol-polyethylene glycol grafted copolymers.
10. Oromucosal delivery system according to embodiment 8 or 9,
wherein the plasticizer is polyethylene glycol or a polyvinyl alcohol-polyethylene glycol grafted copolymer.
11. Oromucosal delivery system according to any one of embodiments 8 to 10,
wherein the active agent-containing layer comprises
   at least 5 wt-%, at least 15 wt-% or at least 20 wt-% of the plasticizer,
   less than or equal to 50 wt-%, less than or equal to 40 wt-%, or less than or equal to 35 wt-% of the plasticizer, and/or
   from 5 to 50 wt-%, from 15 to 40 wt-% or from 20 to 35 wt-% of the plasticizer.
12. Oromucosal delivery system according to embodiment 11,
wherein the active agent-containing layer comprises
   at least 10 wt-%, at least 13 wt-% or at least 15 wt-% of the film-forming agent,
   less than or equal to 75 wt-%, less than or equal to 50 wt-%, or less than or equal to 30 wt-% of the film-forming agent, and/or
   from 10 to 75 wt-%, from 13 to 50 wt-% or from 15 to 30 wt-% of the film-forming agent.
13. Oromucosal delivery system according to any one of embodiments 8 to 12,
wherein the active agent-containing layer comprises a total amount of film-forming agent and plasticizer, wherein said total amount is
   at least 30 wt-%, at least 35 wt-% or at least 40 wt-% of the active agent-containing layer
   less than or equal to 80 wt-%, less than or equal to 60 wt-%, or less than or equal to 45 wt-% of the active agent-containing layer, and/or
   from 30 to 80 wt-%, from 35 to 60 wt-% or from 40 to 45 wt-% of the active agent-containing layer.
14. Oromucosal delivery system according to any one of embodiments 1 to 13,
wherein the film-forming agent is a polymer selected from the group consisting of polyvinyl alcohol, polyvinyl alcohol-polyethylene glycol grafted copolymers, polyethylene oxides, polyvinylpyrrolidone, polyvinyl caprolactam-polyvinyl acetate-polyethylene glycol graft co-polymer, polyethylene glycols, hydroxypropylmethyl cellulose or any mixture thereof
15. Oromucosal delivery system according to embodiment 14,
wherein the film-forming agent is a polyvinyl alcohol.
16. Oromucosal delivery system according to embodiment 15,
wherein the film-forming agent is a polyvinyl alcohol having a molecular weight in the range from 10,000 to 250,000, or a mixture of two or more polyvinyl alcohols, each having a molecular weight in the range from 10,000 to 250,000.
17. Oromucosal delivery system according to any one of embodiments 1 to 7 and 14 to 16,
wherein the active agent-containing layer does not comprise a plasticizer and comprises
   at least 50 wt-%, at least 60 wt-% or at least 65 wt-% of the film-forming agent,
   less than or equal to 85 wt-%, less than or equal to 75 wt-%, or less than or equal to 70 wt-% of the film-forming agent, and/or
   from 50 to 85 wt-%, from 60 to 75 wt-% or from 65 to 70 wt-% of the film-forming agent, and/or
wherein the active agent-containing layer substantially consists of (i) the active agent and (ii) the film-forming agent.
18. Oromucosal delivery system according to any one of embodiments 1 to 17,
wherein the active agent-containing layer further comprises one or more excipients selected from the group consisting of sweeteners, flavoring agents, antioxidants and pH adjusting agents, and/or
wherein the active agent-containing layer does not comprise a pH adjusting agent.
19. Oromucosal delivery system according to embodiment 18,
wherein the sweeteners are selected from the group consisting of sucralose, acesulfame potassium, N—[N-[3-(3-Hydroxy-4-methoxyphenyl)propyl]-α-L-aspartyl]-L-phenylalanin-1-methylester, N—[N-(3,3-Dimethylbutyl)-L-α-aspartyl]-L-phenylalanine 1-methyl ester, aspartame, thaumatine, and/or
wherein the flavoring agents are natural or synthetic flavoring agents such as flavoring compositions selected from the group consisting of a combination of Linalool, Alpha Pinene, Citral, Delta 3 Carene, Beta Pinene and Myrcene, and a combination of Geranyle Acetate, Vanillin, Limonene and Allyl hexanoate.
20. Oromucosal delivery system according to embodiments 19,
wherein the flavoring agent is a combination of Linalool, Alpha Pinene, Citral, Delta 3 Carene, Beta Pinene and Myrcene, or a combination of Geranyle Acetate, Vanillin, Limonene and Allyl hexanoate.
21. Oromucosal delivery system according to embodiment 19,
wherein the sweetener is N—[N-(3,3-Dimethylbutyl)-L-α-aspartyl]-L-phenylalanine 1-methyl ester or N—[N-[3-(3-Hydroxy-4-methoxyphenyl)propyl]-α-L-aspartyl]-L-phenylalanin-1-methylester or a mixture thereof.
22. Oromucosal delivery system according to any one of embodiments 18 to 21,
wherein the active agent-containing layer comprises
   at least 0.05 wt-% or at least 0.5 wt-% of a sweetener,
   less than or equal to 2 wt-% or less than or equal to 1 wt-% of a sweetener, and/or
   from 0.05 wt-% to 2 wt-% or from 0.5 to 1 wt-% of a sweetener.
23. Oromucosal delivery system according to any one of embodiments 18 to 22,
wherein the active agent-containing layer comprises
   at least 0.05 wt-% or at least 0.5 wt-% of a flavoring agent,
   less than or equal to 5 wt-% or less than or equal to 2 wt-% of a flavoring agent, and/or
   from 0.05 wt-% to 5 wt-% or from 0.5 to 2 wt-% of a flavoring agent.
24. Oromucosal delivery system according to any one of embodiments 1 to 23, wherein the oromucosal delivery system does not contain preservatives.
25. Oromucosal delivery system according to any one of embodiments 1 to 24,
   wherein the oromucosal delivery system is in the form of a film.
26. Oromucosal delivery system according to embodiment 25,
   wherein the film has a size of at least 0.5 cm², or less than or equal to 10 cm², or of about 1.5 cm², about 3 cm² or about 6 cm², and/or
   wherein the film has an area weight of at least 100 g/m², at least 110 g/m² or at least 120 g/m² or has an area weight of less than or equal to 400 g/m², less than or equal to 300 g/m² or less than or equal to 250 g/m², and/or
   wherein the film has an area weight from 100 g/m² to less than or equal to 230 g/m² or from 300 g/m² to less than or equal to 400 g/m².
27. Oromucosal delivery system according to any one of embodiments 1 to 26,
wherein the active agent-containing layer is in the form of a flexible foam or a flexible monolithic film.
28. Oromucosal delivery system according to any one of embodiments 1 to 27,
wherein the active agent-containing layer comprises less than or equal to 3 wt-%, less than or equal to 2 wt-%, less than or equal to 1 wt-%, or less than or equal to 0.5 wt-% water.
29. Oromucosal delivery system according to any one of embodiments 1 to 28,
wherein the active agent-containing layer contains initially a total amount of remimazolam-related degradation substances of less than or equal to 0.4 wt-%, less than or equal to 0.3 wt-%, or of less than or equal to 0.2 wt-%, and/or
wherein the active agent-containing layer, when subjected to a storage stability test and after having been stored at 60° C. for up to 6 weeks, contains a total amount of remimazolam-related degradation substances of less than or equal to 0.5 wt-% or of less than or equal to 0.4 wt-%.
30. Oromucosal delivery system according to any one of embodiments 1 to 29,
wherein the active agent-containing layer comprises a plasticizer,
wherein the film-forming agent is a polyvinyl alcohol, and
wherein the ratio of polyvinyl alcohol to the plasticizer is at least 20:80, or is less than or equal to 50:50, or is from 20:80 to 50:50, or is about 25:75 or about 40:60.
31. Oromucosal delivery system according to any one of embodiments 1 to 30,
wherein the oromucosal delivery system is in the form of a film, and
wherein, when a sample film of the oromucosal delivery system with a size of 5.75 cm² is dissolved in 5 mL of artificial saliva or of 0.9% NaCl solution, the pH of the resulting solution as measured by a pH electrode ranges from pH 3.0 to pH 3.7.
32. Oromucosal delivery system according to any one of embodiments 1 to 31, wherein the oromucosal delivery system provides, after single administration to the buccal mucosa of the oral cavity of a human subject,
   a bioavailability of greater than 10% based on cₘₐₓ or of greater than 20% based on AUC,
   a cₘₐₓ of equal to or greater than 0.3 μg/mL, and/or
   a tₘₐₓ of 10 to 40 min, preferably 10 to 20 min.
33. Oromucosal delivery system according to any one of embodiments 1 to 32 for use in producing sedation, producing hypnosis, producing anxiolysis, producing muscle relaxation, the treatment of convulsions or the induction of amnesia for perioperative events.
34. Oromucosal delivery system for use in sedation according to embodiment 33,
wherein sedation is procedural sedation such as sedation for dental procedures or sedation for diagnostic procedures, preoperative sedation, and/or conscious sedation.
35. Oromucosal delivery system for use in sedation according to embodiment 33 or 34,
wherein sedation is induced before and/or during endoscopy, colonoscopy, or other diagnostic or operative procedures.
36. Oromucosal delivery system for use in sedation according to any one of embodiments 33 to 35,
wherein mild sedation, moderate sedation, deep sedation or general anesthesia is reached, which preferably has a duration of 5 to 30 minutes, 8 to 20 minutes or 10 to 15 minutes.
37. Oromucosal delivery system for use in sedation according to any one of embodiments 33 to 36,
wherein the oromucosal delivery system is administered by applying the active agent-containing layer to the mucosa, and in particular to the buccal, sublingual, gingival or palatal mucosa, of the oral cavity of a human patient and maintained on the mucosa until dissolved.
38. A method of producing sedation, producing hypnosis, producing anxiolysis, producing muscle relaxation, treating convulsions or inducing amnesia for perioperative events,
wherein the oromucosal delivery system according to any one of embodiments 1 to 32 is administered to a subject.
39. Method of sedation according to embodiment 38,
wherein sedation is procedural sedation, preoperative sedation, sedation for dental procedures, sedation for diagnostic procedures, and/or conscious sedation.
40. Method of sedation according to embodiment 38 or 39,
wherein sedation is induced before and/or during endoscopy, colonoscopy, or other diagnostic or operative procedures.
41. Method of sedation according to any one of embodiments 38 to 40,
wherein mild sedation, moderate sedation, deep sedation or general anesthesia is reached, which preferably has a duration of 5 to 30 minutes, 8 to 20 minutes or 10 to 15 minutes.
42. Method of sedation according to any one of embodiments 38 to 41,
wherein the oromucosal delivery system is administered by applying the active agent-containing layer to the mucosa, and in particular to the buccal, sublingual, gingival or palatal mucosa, of the oral cavity of a human patient and maintained on the mucosa until dissolved.
43. Use of an oromucosal delivery system according to any one of embodiments 1 to 32 in the preparation of a medicament for producing sedation, producing hypnosis, producing anxiolysis, producing muscle relaxation, the treatment of convulsions or the induction of amnesia for perioperative events.
44. Use of an oromucosal delivery system in the preparation of a medicament for producing sedation according to embodiment 43,
wherein sedation is procedural sedation, preoperative sedation, sedation for dental procedures, sedation for diagnostic procedures, and/or conscious sedation.
45. Use of an oromucosal delivery system in the preparation of a medicament for producing sedation according to embodiment 43 or 44,
wherein sedation is induced before and/or during endoscopy, colonoscopy, or other diagnostic or operative procedures.
46. Use of an oromucosal delivery system in the preparation of a medicament for producing sedation according to any one of embodiments 43 to 45,
wherein mild sedation, moderate sedation, deep sedation or general anesthesia is reached, which preferably has a duration of 5 to 30 minutes, 8 to 20 minutes or 10 to 15 minutes.
47. A pharmaceutical product comprising
a packaging, and
one or more unit doses of the oromucosal delivery system according to any one of embodiments 1 to 32.
48. Pharmaceutical product according to embodiment 47,
wherein the packaging is in the form of a pouch.
49. Pharmaceutical product according to embodiment 48,
wherein the pouch is made of a multi-layer film material comprising an outward-facing paper layer, an intermediate polyethylene layer and an inward-facing aluminum layer, and/or wherein the pouch is sealed with a sealant selected from the group consisting of an ethylene copolymer, a polyethylene terephthalate copolymer and a cyclic olefin copolymer.
50. Pharmaceutical product according to embodiment 48 or 49,
wherein the pouch comprises one or more desiccant(s) or does not comprise a desiccant.
51. Pharmaceutical product according to embodiment 50,
wherein the pouch comprises one or more desiccant(s) and the desiccant comprises silica gel, molecular sieves 4 Å and/or zeolite molecular sieves 4 Å as a drying agent, and/or wherein the desiccant is in the form of an adhesive film.
52. Pharmaceutical product according to any one of embodiments 47 to 51,
further comprising a folded polyethylene terephthalate foil, wherein the one or more unit dose(s) of the oromucosal delivery system are enclosed by the folded polyethylene terephthalate foil which is folded around and protects the unit dose(s) from further contact with the packaging.
53. Pharmaceutical product according to embodiment 51 or 52,
wherein the pouch comprises one or more desiccant(s) in the form of an adhesive film, which is attached on the inward-facing side of the pouch, or, where present, the outward-facing side of the folded polyethylene terephthalate foil so that contact with the oromucosal delivery system is avoided.
54. Pharmaceutical product according to any one of embodiments 47 to 53,
wherein the pouch is nitrogen-filled.
55. Process of manufacture of an active agent-containing layer as defined in any one of embodiments 1 to 32 comprising the steps of:
   i. combining at least (i) remimazolam, a pharmaceutically acceptable salt, or any other form thereof, as active agent, and (ii) a film-forming agent to obtain a mixture; and
   ii. forming the active agent-containing layer.
56. Process of manufacture according to embodiment 55,
wherein the process is a hot-melt process, which is
   either
a hot-melt extrusion process comprising step ii. of:
   a. introducing the mixture comprising the active agent and the film-forming agent with or without further excipients into an extruder;
   b. heating said mixture to at least the softening temperature of said mixture; and
   c. extruding the heated mixture comprising the film-forming agent and the active agent in the form of a film to obtain the active agent-containing layer,
   or
a vacuum compression molding process comprising step ii. of
   a. introducing the mixture comprising the active agent and the film-forming agent with or without excipients into a sample chamber; and
   b. compacting said mixture while applying vacuum and heating said mixture to at least the softening temperature of said mixture to obtain the active agent-containing layer.
57. Process of manufacture according to embodiment 55,
wherein the process is a coating process comprising the steps of:
   i. dispersing or dissolving the active agent in a solution of the film-forming agent with or without further excipients to obtain a coating composition, and
   ii. a. coating the coating composition on a coating substrate; and
      b. drying the laminate segments in a drying oven to obtain the active agent-containing layer in the form of a flexible monolithic film.
58. Process of manufacture according to embodiment 55,
wherein the process is a foam forming process comprising the steps of:
   i. dispersing or dissolving the active agent in an aqueous solution of the film-forming agent with or without further excipients to obtain a coating composition, and
   ii. a. foaming the coating composition to obtain a foam coating composition;
      b. coating the foam coating composition on a coating substrate; and
      c. drying the laminate segments in a drying oven to obtain the active agent-containing layer in the form of a foam.
59. Process of manufacture according to embodiment 58,
wherein foaming is conducted by stirring while introducing nitrogen gas into the composition.
60. Process of manufacture of an oromucosal delivery system comprising an active agent-containing layer comprising (i) remimazolam, a pharmaceutically acceptable salt, or any other form thereof, as active agent, and (ii) a film-forming agent according to any one of embodiments 1 to 32 comprising the steps of:
   i. combining at least the active agent and a film-forming agent to obtain a mixture; and
   ii. forming the active agent-containing layer
as defined in any one of embodiments 50 to 59.
61. Oromucosal delivery system obtainable by a process according to embodiment 60.
62. Oromucosal delivery system according to embodiment 1, wherein the active agent-containing layer comprises
   i) 55 to 60 wt-% of remimazolam besylate;
   ii) 10 to 15 wt-% of polyvinyl alcohol as film-forming agent,
   iii) 30 to 35 wt-% of a polyvinyl alcohol-polyethylene glycol grafted copolymer,
   iv) from 0.05 to 1 wt-% of one or more sweeteners, and
   v) from 0.5 to 2 wt-% of a flavoring agent
wherein
the area weight of the active agent-containing layer is less than or equal to 200 g/m².
63. Oromucosal delivery system according to embodiment 1, wherein the active agent-containing layer comprises
   i) 55 to 60 wt-% of remimazolam besylate;
   ii) 15 to 20 wt-% of polyvinyl alcohol as film-forming agent
   iii) 20 to 26 wt-% of a polyvinyl alcohol-polyethylene glycol grafted copolymer,
   iv) from 0.05 to 1 wt-% of one or more sweeteners, and
   v) from 0.5 to 2 wt-% of a flavoring agent
wherein
the area weight of the active agent-containing layer is less than or equal to 200 g/m².

1. Oromucosal delivery system for the transmucosal delivery of an active agent, comprising
an active agent-containing layer comprising:
i) remimazolam, a pharmaceutically acceptable salt, or any other form thereof, as active agent and
ii) a film-forming agent,
wherein the oromucosal delivery system is in the form of a film.

2. Oromucosal delivery system according to claim 1,
wherein the active agent-containing layer comprises
at least 20 wt-%, at least 25 wt-% or at least 30 wt-% of the active agent, and/or
less than or equal to 60 wt-%, less than or equal to 55 wt-%, or less than or equal to 50 wt-% of the active agent, and/or
from 20 to 60 wt-%, from 25 to 55 wt-% or from 30 to 50 wt-% of the active agent.

3. Oromucosal delivery system according to claim 1 or 2,
wherein the active agent is remimazolam besylate or remimazolam tosylate.

4. Oromucosal delivery system according to any one of claims 1 to 3 comprising an active agent-containing layer comprising:
i) remimazolam, a pharmaceutically acceptable salt, or any other form thereof, as active agent,
ii) a film-forming agent, and
iii) a plasticizer.

5. Oromucosal delivery system according to any one of claims 1 to 4,
wherein the plasticizer is polyethylene glycol or a polyvinyl alcohol-polyethylene glycol grafted copolymer, and/or
wherein the film-forming agent is a polymer selected from the group consisting of polyvinyl alcohol, polyvinyl alcohol-polyethylene glycol grafted copolymers, polyethylene oxides, polyvinylpyrrolidone, polyvinyl caprolactam-polyvinyl acetate-polyethylene glycol graft co-polymer, polyethylene glycols, hydroxypropylmethyl cellulose or any mixture thereof.

6. Oromucosal delivery system according to any one of claims 1 to 5,
wherein the film-forming agent is a polyvinyl alcohol having a molecular weight in the range from 10,000 to 250,000, or a mixture of two or more polyvinyl alcohols, each having a molecular weight in the range from 10,000 to 250,000.

7. Oromucosal delivery system according to any one of claims 1 to 6,
wherein the active agent-containing layer further comprises one or more excipients selected from the group consisting of sweeteners, flavoring agents, antioxidants and pH adjusting agents, and/or
wherein the active agent-containing layer does not comprise a pH adjusting agent.

8. Oromucosal delivery system according to claim 7,
wherein the sweeteners are selected from the group consisting of sucralose, acesulfame potassium, N—[N-[3-(3-Hydroxy-4-methoxyphenyl)propyl]-α-L-aspartyl]-L-phenylalanin-1-methylester, N—[N-(3,3-Dimethylbutyl)-L-α-aspartyl]-L-phenylalanine 1-methyl ester, aspartame, thaumatine, and/or
wherein the flavoring agents are natural or synthetic flavoring agents such as flavoring compositions selected from the group consisting of a combination of Linalool, Alpha Pinene, Citral, Delta 3 Carene, Beta Pinene and Myrcene, and a combination of Geranyle Acetate, Vanillin, Limonene and Allyl hexanoate.

9. Oromucosal delivery system according to any one of claims 1 to 8,
wherein the active agent-containing layer is in the form of a flexible foam or a flexible monolithic film.

10. Oromucosal delivery system according to any one of claims 1 to 9,
wherein the oromucosal delivery system is in the form of a film, and
wherein, when a sample film of the oromucosal delivery system with a size of 5.75 cm² is dissolved in 5 mL of artificial saliva or of 0.9% NaCl solution, the pH of the resulting solution as measured by a pH electrode ranges from pH 3.0 to pH 3.7.

11. Oromucosal delivery system according to any one of claims 1 to 10, wherein the oromucosal delivery system provides, after single administration to the buccal mucosa of the oral cavity of a human subject,
a bioavailability of greater than 10% based on cₘₐₓ or of greater than 20% based on AUC,
a cₘₐₓ of equal to or greater than 0.3 μg/mL, and/or
a tₘₐₓ of 10 to 40 min or of 10 to 20 min.

12. Oromucosal delivery system according to any one of claims 1 to 11 for use in producing sedation, producing hypnosis, producing anxiolysis, producing muscle relaxation, the treatment of convulsions or the induction of amnesia for perioperative events.

13. A pharmaceutical product comprising
a packaging, and
one or more unit doses of the oromucosal delivery system according to any one of claims 1 to 11.

14. Pharmaceutical product according to claim 13,
wherein the packaging is in the form of a pouch,
wherein the pouch comprises one or more desiccant(s).

15. Process of manufacture of an active agent-containing layer as defined in any one of claims 1 to 11 comprising the steps of:
i. combining at least (i) remimazolam, a pharmaceutically acceptable salt, or any other form thereof, as active agent, and (ii) a film-forming agent to obtain a mixture; and
ii. forming the active agent-containing layer.

16. Process of manufacture according to claim 15,
wherein the process is a foam forming process comprising the steps of:
i. dispersing or dissolving the active agent in an aqueous solution of the film-forming agent with or without further excipients to obtain a coating composition, and
ii. a. foaming the coating composition to obtain a foam coating composition;
b. coating the foam coating composition on a coating substrate; and
c. drying the laminate segments in a drying oven to obtain the active agent-containing layer in the form of a foam.

17. Process of manufacture of an oromucosal delivery system comprising an active agent-containing layer comprising (i) remimazolam, a pharmaceutically acceptable salt, or any other form thereof, as active agent, and (ii) a film-forming agent according to any one of claims 1 to 11 comprising the steps of:
i. combining at least the active agent and a film-forming agent to obtain a mixture; and
ii. forming the active agent-containing layer
as defined in claim 15 or 16.

18. Oromucosal delivery system obtainable by a process according to claim 17.

19. Oromucosal delivery system according to claim 1, wherein the active agent-containing layer comprises
i) 55 to 60 wt-% of remimazolam besylate;
ii) 10 to 15 wt-% of polyvinyl alcohol as film-forming agent,
iii) 30 to 35 wt-% of a polyvinyl alcohol-polyethylene glycol grafted copolymer,
iv) from 0.05 to 1 wt-% of one or more sweeteners, and
v) from 0.5 to 2 wt-% of a flavoring agent
wherein
the area weight of the active agent-containing layer is less than or equal to 200 g/m².

20. Oromucosal delivery system according to claim 1, wherein the active agent-containing layer comprises
i) 55 to 60 wt-% of remimazolam besylate;
ii) 15 to 20 wt-% of polyvinyl alcohol as film-forming agent
iii) 20 to 26 wt-% of a polyvinyl alcohol-polyethylene glycol grafted copolymer,
iv) from 0.05 to 1 wt-% of one or more sweeteners, and
v) from 0.5 to 2 wt-% of a flavoring agent
wherein
the area weight of the active agent-containing layer is less than or equal to 200 g/m².
